(19) United States
(12) Patent Application Publication
(10) Pub. No.: US 2010/0042137 A1
(43) Pub. Date: Feb. 18, 2010

(54) ACUPUNCTURE AND ACUPRESSURE THERAPIES

(76) Inventors: Bryan T. ORONSKY, Los Altos Hills, CA (US); Neil C. Oronsky, Los Altos Hills, CA (US); Arnold L. Oronsky, Los Altos Hills, CA (US)

Correspondence Address: MORRISON & FOERSTER LLP, 755 PAGE MILL RD, PALO ALTO, CA, 94304-1018, US

(21) Appl. No.: 12/389,248

(22) Filed: Feb. 19, 2009

Related U.S. Application Data
(60) Provisional application No. 61/066,327, filed on Feb. 19, 2008.
(60) Provisional application No. 61/061,532, filed on Jun. 13, 2008.

(51) Int. Cl. A61B 17/00; A61M 37/00; A61M 5/00; A61M 31/00

(52) U.S. Cl. 606/204; 604/141; 604/112; 604/500

(57) ABSTRACT

Described here are devices, methods, and kits for acupuncture or acupressure therapy. The devices may include a tissue-piercing member and a reservoir containing an agent. The devices and agents may be packaged as kits useful for relieving pain and/or treating various medical conditions. The devices, methods, and kits may enable less skilled practitioners to administer acupuncture or acupressure therapy, and may extend the duration of pain relief or other symptom relief.

### CROSS-REFERENCE TO RELATED APPLICATIONS

This application claims priority to U.S. Provisional Application Ser. No. 61/066,327 filed on Feb. 19, 2008, and U.S. Provisional Application Ser. No. 61/061,532 filed on Jun. 13, 2008, each of which is hereby incorporated by reference in its entirety.

### FIELD

Described here are devices for administering acupuncture and acupressure therapies. Specifically, devices that deliver an agent to, or adjacent to, an acupuncture point are described. Methods and kits including the devices are also described.

### BACKGROUND

Eastern medicine views health and wellness in terms Qi or internal energy which flows along specific channels or meridians. When this flow is blocked, the result is dysfunction or disease. Pain is viewed as one manifestation of an imbalance or blockage in the flow of Qi. In Western medicine, the concept of disease is more mechanistic, based on a macroscopic or microscopic dysfunction that can be measured, quantified, and objectified. Thus, under a Western view, pain is a subjective experience that results from a complex series of mechanisms that involve the firing of nociception, touch, and pressure receptors in the periphery, and subsequent transmission to the spinal cord and to lower and higher centers of the brain with the possibility of modification in a multitude of ways at each level of the pain pathway. See, e.g., Millan, M. J. (1999), The Induction of Pain: An Integrative Review, Progress in Neurobiology, 57, 1-164 (Pergamon Press) for an in-depth review.

Western medicine generally treats medical conditions using available therapeutic agents. However, as complementary and alternative medicine (CAM) therapies such as herbal therapy, acupuncture, acupressure, yoga, chiropractic, relaxation techniques, nutrition, and dietary supplements continue to gain popularity, there is increasing pressure on clinicians to integrate a repertoire of Eastern and Western approaches.

Acupuncture therapy is an Eastern medical treatment used to alleviate pain and treat various medical conditions. In acupuncture therapy, the movement of Qi is altered by inserting sharp, thin needles into locations on the body where the Qi is believed to flow. These locations are known as acupuncture points. Typically, each acupuncture point is known to affect one or more specific body parts, organs, types of pain, or medical conditions. In acupuncture therapy, it is believed that there are twelve main meridians, eight secondary meridians, and more than 2,000 acupuncture points on the human body that connect with them. It is also believed that the underlying analgesic mechanism of acupuncture is due to the release of pain-killing substances such as endorphins and other substances by immune system cells at specific sites in the body. In addition, studies have shown that acupuncture may alter brain chemistry by influencing the release of neurotransmitters and neurohormones. These neurotransmitters and neurohormones in turn may affect the parts of the central nervous system related to sensation and involuntary body functions, such as immune reactions and processes that regulate an individual's blood pressure, blood flow, and body temperature (http://nccam.nih.gov/health/acupuncture/#how).

Some of the most “influential points” on the body are named accordingly. Each influential point is believed to be associated with a specific effect on a particular tissue, body area, or organ system. For example, the stimulation of GB 34 is thought to have a positive effect on any symptom dealing with muscles, ligaments, or tendons. GB 39 is thought to be the “influential point of marrow,” and is located just above the external malleolus. GB 39 is believed to have a particular effect on both sciatic neuritis and cervicalgia, as it is a specific point linking the yang meridians of the lower extremity, namely the gallbladder, stomach, and bladder. This point has also been used to treat vertigo and brain dysfunction. LU 9 is believed to be the “influential point of the vessels,” a point often used to help make the pulse more prominent in those with fine and weak pulses. For a complete review of influential points, see Amaro, John A. The Eight (Hui) Influential Points. Dynamic Chiropractic 18:7 (2000).

Acupressure therapy is based on the principles of acupuncture therapy. In acupressure therapy, pressure or some other stimulus is applied to the surface of the skin at one or more acupuncture points to alleviate pain and/or treat a medical condition. The stimulus may include light, heat, sound waves, electricity, or the like. Generally, the stimulus is applied to the same locations on the body that are used in acupuncture therapy.

While acupuncture therapy and acupressure therapy provide some benefit in terms of pain relief or alleviation of a medical condition, this benefit is often short-lived and frequent treatments are required. Further, only very skilled practitioners are able to accurately and precisely locate acupuncture points on the body.

Accordingly, an acupuncture therapy that can be administered by less skilled practitioners would be useful. Kits that can be used by the practitioners or by the patients themselves in providing acupuncture therapy would also be desirable. In particular, an easy to administer acupuncture therapy that extends the period of time during which relief is felt without compromising the effectiveness of the therapy would be desirable.

### SUMMARY

Described here are devices, methods, and kits for acupuncture therapy. The described devices, methods, and kits may extend the period of time during which relief is felt and/or may not require an expert practitioner to locate an acupuncture point. The methods integrate Eastern and Western approaches, by employing the modality of injection to acupuncture points and the philosophy of acupuncture to soft tissue injection. It is yet another objective to inject, implant, or infuse a plurality of points over the body for therapeutic effect—not only those points recognized by traditional acupuncture but also those overlying inflamed or damaged joints, bursae, tendons, nerves, muscles, ligaments, tendons, entheses and other soft tissue structures according to a traditional Western approach intradermally, i.e., at a much shallower depth. In another aspect, elements of acupuncture and acupressure are combined by combining needle puncture with the injection of a volumetric fluid that exerts pressure.

A method is also described for treating pain and promoting health that involves selecting a point of injection or implantation of an agent, and injecting, infusing or implanting a device or an agent formulation at an acupuncture point or an area adjacent thereto. Pain relief or relief from a non-painful condition may be obtained within minutes and lasts for periods of variable duration ranging from minutes to several hours and even, in some cases, days. The method of treatment may be effective to treat visceral, somatic, inflammatory, post-surgical and neuropathic pain both acute and chronic as well as muscle pain and stiffness and joint pain and stiffness locally and/or systemically and to promote health and wellness even in non-painful conditions. Examples demonstrate pain relief in human patients for a wide number of conditions, including joint, muscle and tendon pain, joint, muscle and tendon immobility, inflammatory pain, post-surgical pain, headaches, neuropathies, osteoarthritis and autoimmune disorders and promotion of wellness in non-painful conditions such as sialorrhea, nausea, hypersomnia, allergies, and mood and sleep disturbances.

Various devices may be used to deliver an agent topically or subcutaneously. Some of the devices for acupuncture therapy described here generally comprise a tissue-piercing member and a reservoir. The tissue-piercing member is typically used to deliver an agent subcutaneously. The reservoir may contain the agent. The device may include additional elements. For example, the device may further comprise a housing. The housing typically supports a proximal end of the tissue-piercing member and the reservoir. In some variations, the housing may deliver a predetermined amount of the agent from the reservoir to the tissue-piercing member. The tissue-piercing member may be uniformly rigid or include a malleable portion. The tissue-piercing member may comprise an acupuncture needle. The tissue-piercing member may comprise a lumen and an interior surface of the lumen may be coated with the agent. The reservoir can have various configurations. For example, a distal end of the reservoir may comprise a tissue-piercing member. The reservoir may be removably coupled to the tissue-piercing member. The reservoir may be frangible. The agent contained within the reservoir may include a single agent or a combination of agents. In some variations, the agent may comprise a local anesthetic such as lidocaine. The device may comprise more than one reservoir. A second reservoir may contain a second agent that can be mixed with, or delivered separately from, the agent in a first reservoir. The second agent may be delivered using the same tissue-piercing member or a different tissue-piercing member. The device may comprise a pump configured to deliver the agent. The pump may deliver the agent periodically, continuously, or based on a user input.

Other devices for acupuncture therapy described here comprise a tissue-piercing member that delivers an agent when it is advanced into tissue at or adjacent to an acupuncture point. The tissue-piercing member may comprise an acupuncture needle or may include one or more lumens. In some variations, the tissue-piercing member is coated or impregnated with the agent. For example, the tissue-piercing member may be spray-coated or dip-coated with the agent.

Methods for performing acupuncture are also described here. In some variations, the methods comprise inserting a tissue-piercing member into tissue at or adjacent to an acupuncture point and delivering an agent into the tissue. The agent may be delivered in any suitable manner. For example, some of the methods comprise pre-treating the tissue by topically administering an agent to the surface of the skin at an acupuncture point or an acupressure point. These methods may further include passing the tissue-piercing member through the agent at the tissue. According to some embodiments, the tissue-piercing member is coated with the agent prior to insertion into the tissue. The tissue-piercing member may be coupled to an implantable pump in fluid communication with a reservoir. The agent may be delivered using a syringe coupled to the tissue-piercing member. The tissue-piercing member may comprise an acupuncture needle.

The agent for use with the devices, methods, and kits described here may be any suitable agent and be in any suitable form. For example, the agent may be a liquid, a solid, or a semisolid. The liquid may be a solution or an emulsion. The solid may be a powder, a suppository, or a patch. The semi-solid may be a cream, a lotion, an ointment, or a gel. The agent may be configured for sustained release. The agent may be used to treat a joint condition such as inflammatory arthropathy, bursitis, tendinopathy, sprains, arthralgias, osteoarthritis, degenerative joint disease, spondylosis, TMJ dysfunction, or fibromyalgia. The agent may used to treat a muscular condition such as muscle stiffness, overuse syndrome, or muscle strains. The agent may be used to treat a respiratory condition such as asthma, atelectasis, chronic obstructive pulmonary disease, wheezing, or dyspnea. The agent may be used to treat a circulatory condition such as high blood pressure or headache. The agent may be used to treat a nervous condition such as neuropathy, polyneuropathy, tension headache, headache, mood disturbance, sleep disturbance, fatigue, hypersomnia, or mood disorder. The agent may be used to treat an endocrinal condition such as pancreatitis, diabetes, or allergy. The agent may be used to treat other conditions such as, but not limited to, obesity, pain, chest tightness, testicular torsion, sialorrhea, indigestion, ulcers, fracture or compression of lumbar vertebrae, or reflex sympathetic dystrophy.

The agent may comprise anti-atherosclerotic agents, anti-psoriatics, antispasmodics, muscle relaxants, muscle contractants, histamines, antipyretics, analgesics, antihypertensives, anticoagulants, procoagulants, cholesterol-reducing agents, anticonvulsants, cognitive enhancers, cholinergics, anti-cholinergics, anti-Alzheimer's disease agents, sedatives, anti-Parkinson substances, hypnotics, anti-psychotic substances, antacids, antihistamines, antidiabetics, contraceptives, sympathomimetics, coenzymes, adrenergics, adrenergic antagonists, enzyme inhibitors, neurotoxins, neurotransmitters, hormones, anti-ulcer agents, antiflatulents, proton pump inhibitors, antidiarrheals, antipruritics, anti-emetics, antireflux agents, antiobesity agents, autoimmune disorder agents, anti-cancer substances, immunomodulatory factors, diuretics, anti-glaucoma compounds, anti-inflammatory agents, anti-vertigo medications, local anesthetics, ophthalmics, trophic factors, growth factors, nucleic acids, anti-infectives, vitamins, minerals, nutritional supplements, lubricants, imaging agents, emulsifying stabilizers, herbs, plant extracts, astringents, bupivicaine, triamcinolon, combinations, derivatives, or precursors thereof.

Also described are other methods for acupuncture therapy performed using an implantable reservoir or device. These methods generally comprise subcutaneously inserting an implantable reservoir at or adjacent to an acupuncture point. The implantable reservoir may contain an agent for treating a medical condition. The implantable reservoir may comprise a wick.

Also described are patches to be applied to the surface of the skin. A patch for acupuncture therapy may comprise a plurality of tissue-piercing members and an agent in fluid communication with the tissue-piercing members. The plurality of tissue-piercing members may be configured to deliver the agent topically or subcutaneously. The patch may adhere to skin for an extended period of time. To adhere to the skin, the patch may include one or more suitable adhesives.

Kits comprising one or more devices for acupuncture therapy and one or more reservoirs for one or more agents are also described. The kit may comprise instructions for use that may include instructions for selecting an acupuncture point based on a type of pain. The one or more devices for acupuncture therapy may be coated with the one or more agents. The reservoir may comprise at least one blister containing the one or more agents.

### BRIEF DESCRIPTION OF THE DRAWINGS

FIG. 1 depicts an exemplary acupuncture needle coated with an agent.

FIG. 2 depicts an exemplary dual lumen needle for use with an acupuncture needle.

FIG. 3 shows an exemplary injection device for use with an acupuncture needle.

FIG. 4 shows an exemplary implantable reservoir.

FIG. 5 illustrates an exemplary patch that can be applied to the surface of the skin.

### DETAILED DESCRIPTION

Described here are devices for acupuncture or acupressure therapy. The devices may be configured in any suitable manner and may include any suitable agent for relieving pain or treating a medical condition. For example, the devices may be configured as implants, tissue-piercing members, or topically applied devices, e.g., patches. When an agent is used with the device, the agent may be provided in any dosage form. For example, the agent may be formulated as a solid, semi-solid, liquid, depot for sustained or controlled release, etc. Some devices may be coated with the agent formulation. In other variations, the agent formulation is applied to an area of skin and then the device placed on or through the skin within that area, or the device implanted underneath the skin of that area. In variations where a device is not used, the agent formulation may be injected into any skin layer at an acupuncture point. The devices and/or agent formulations may have a therapeutic effect when placed or implanted at an acupuncture point or adjacent to an acupuncture point.

When an agent is used in combination with the devices described herein, the inventors have surprisingly observed additional benefits to acupuncture therapy. A first benefit is that the combination therapy may be effective at treating the medical condition or relieving pain for a longer period of time than the acupuncture therapy alone. For example, some combination therapies effectively treat pain for months rather than weeks. A second surprising benefit is that the devices need not be placed or inserted exactly at the acupuncture point in order to be effective. Here this would allow patients who might not be able to remain completely stationary through an acupuncture therapy session to receive treatment. Further, by eliminating the need to locate the exact location of an acupuncture point, medical practitioners who do not specialize in acupuncture therapy can use the devices to treat individuals.

### I. DEVICES

The devices described here may be used to more effectively provide acupuncture and acupressure therapies. As previously stated, the devices may be placed at an acupuncture point or an area adjacent thereto. Injections or infusions of an agent may also be administered in this manner. The agent may be administered to relieve pain and/or treat a medical condition.

In some variations, the device is configured as a depot that releases one or more agents in a controlled release, sustained release, extended release, or delayed release fashion. For example, the depot may be a polymer matrix loaded with one or more agents, which are subsequently released, e.g., by dissolution or diffusion into the surrounding environment, or by polymer degradation. The depot may comprise any suitable biocompatible material, which may or may not be polymeric. When a polymeric material is employed, the polymers may be biodegradable or nonbiodegradable.

Exemplary biodegradable polymers for use in the depots described here include without limitation, alginate, cellulose and ester, collagen, dextran, elastin, fibrin, polysaccharides, hyaluronic acid, polyacetal, polyacrylates (L-tyrosine-derived or free acid), poly(β-hydroxyesters), polyamides, poly(amino acid), polyalkanotes, polyalkylene alkylates, polyalkylene oxylates, polyalkylene succinates, polyanhydrides, polyanhydride esters, polyaspartimic acid, polylactic acid, polybutylene digloclate, poly(caprolactone), poly(caprolactone)/poly(ethylene glycol) copolymers, polycarbone, L-tyrosin-derived polycarbonates, polycyanoacrylates, polydihydropyrans, poly(dioxanone), poly-p-dioxanone, poly(ε-caprolactone-dimethyltrimethylene carbonate), poly(esteramide), polyesters, aliphatic polyesters, poly(etherester), polyethylene glycol/poly(orthoester) copolymers, poly(glutarunic acid), poly(glycolic acid), poly(glycolide), poly(glycolide)/poly(ethylene glycol) copolymers, poly(lactide), poly(lactide-co-caprolactone), poly(DL-lactide-co-glycolide), poly(lactide-co-glycolide)/poly(ethylene glycol) copolymers, poly(lactide)poly(ethylene glycol) copolymers, polypeptides, polyphosphazenes, polyphosphesters, polyphophoester urethanes, poly(propylene fumarate-co-ethylene glycol), poly(trimethylene carbone), polytyrosine carbonate, polyurethane, PorLastin or silk-elastin polymers, spider silk, tephaflex, terpolymer (copolymers of glycolide lactide or dimethyltrimethylene carbonate), and combinations, mixtures, copolymers, and blends thereof.

If a nonbiodegradable polymer is used to make or incorporate into the depot, suitable nonbiodegradable polymers include, but are not limited to, poly(ethylene vinyl acetate), poly(vinyl acetate), silicone polymers, polyurethanes, polysaccharides such as a cellulosic polymers and cellulose derivatives, acyl substituted cellulose acetates and derivatives thereof, copolymers of poly(ethylene glycol) and poly(butylene terephthalate), polystyrenes, polyvinyl chloride, polyvinyl fluoride, poly(vinyl imidazole), chorosulphonated polyolefins, polyethylene oxide, and combinations, mixtures, copolymers, and blends thereof.

In other variations, and as further described below, the device is configured so that a tissue-piercing member may be used to deliver an agent to the acupuncture point or an area adjacent thereto. The agent and tissue-piercing member may be configured as an integrated unit, e.g., a needle coated with the agent, or as modular units within a device, e.g., an array of microneedles that deliver an agent.

In some variations, the tissue-piercing members are solid rods or needles. In other variations, the tissue-piercing members comprise one or more lumens extending therethrough. The tissue-piercing members are generally configured to have a small diameter. For example, they may have gauge ranging from about 28 to about 40. The tissue-piercing members may also have any suitable length. For example, the tissue-piercing members may be from about 0.04 cm to about 4 cm in length. For example, the tissue-piercing members may be about 3.8 cm (about 1.5 inches), about 1.6 cm (⅝ inch), or about 1.3 cm (0.5 inches) in length. In some instances, it may be beneficial to have a length of about 3 cm so that insertion just below the dermal layer can be achieved. The tissue-piercing members may be uniformly rigid or include a malleable portion. In some variations, the tissue-piercing members are configured with one or more coatings of an agent. For example, an agent may be provided in a coating on the exterior surface of the tissue-piercing member or a luminal surface of the tissue-piercing member. The coating may be formed by spray-coating or dip-coating the appropriate tissue-piercing member surface with the agent. In some variations, the tissue-piercing member is impregnated with the agent.

In further variations, the devices are configured to include a housing for holding at least a proximal end of the tissue-piercing member and a reservoir with an agent contained therein. Some variations of the housing may additionally enclose the distal end of the tissue-piercing member when the device is not in use. The housing may also include a mechanism to deliver a predetermined amount of the agent from the reservoir to the tissue-piercing member. In other variations, a sponge-like material may be placed over the distal end and/or tip of the tissue-piercing member. Any suitable sponge-like material may be used. For example, natural sponge materials (e.g., collagen and other organic materials) and synthetic sponge materials may be used. When synthetic sponge-materials are employed, materials including, but not limited to, foam (e.g., open or closed cell foam), low-density polyether, polyester, and combinations thereof, may be used. The sponge-like material may have any suitable length, width, thickness, and geometry. The sponge-like material may be used to absorb a fluid, e.g., an agent in liquid form, which can then be discharged when pressure is applied, e.g., when pressure is applied to the tissue-piercing member to puncture the skin.

The reservoir may have any suitable configuration. For example, a distal end of the reservoir may comprise a tissue-piercing member. The reservoir may be removably coupled to the tissue-piercing member. The reservoir may be frangible. The agent contained within the reservoir may include a single agent or a combination of agents. In some variations, the agent may comprise a local anesthetic such as lidocaine. In other variations, two or more reservoirs may be included in the devices. Here the second reservoir may contain a second agent that can be mixed with, or delivered separately from, the agent in the first reservoir. The second agent may be delivered using the same tissue-piercing member or a different tissue-piercing member. The device may also comprise a pump configured to deliver the agent. The pump may deliver the agent periodically, continuously, or based on user input.

Tissue-Piercing Members

The tissue-piercing members may be of any configuration. For example, they may comprise a solid or hollow rod. The hollow rods may include a single lumen or multiple lumens. The tissue-piercing members may also have any suitable length for delivering the agent subcutaneously, e.g., into the epidermis, dermis, or hypodermis. In some variations, the tissue-piercing members may be advanced to a relatively shallow depth, e.g., into the epidermis. In this instance, the devices may result in reduced pain or blood loss at the injection site. Further, by not advancing beyond the epidermis, the risk of infection, nerve damage, and the spread of blood-borne diseases may be reduced. In other variations, the tissue-piercing members may be advanced into the dermis or the hypodermis. For example, the tissue-piercing members may be configured to advance between about 2 mm to about 6 mm into the skin.

The tissue-piercing members are generally configured with a small diameter in order to reduce pain on insertion into the skin. For example, the tissue-piercing members may be small gauge needles, e.g., about 28 gauge, about 30 gauge, about 32 gauge, about 34 gauge, about 36 gauge, about 38 gauge, or about 40 gauge acupuncture needles. In some variations, microneedles may be used. The microneedles may be made from any suitable material including, but not limited to, ceramic, glass, and metals, e.g., silicon, stainless steel, and alloys thereof. In some variations, a plurality of microneedles may be used in connection with a syringe pump and/or a patch, as further described below. A portion of the tissue-piercing member may be malleable. In one variation, the tissue-piercing member is included in an injection pen.

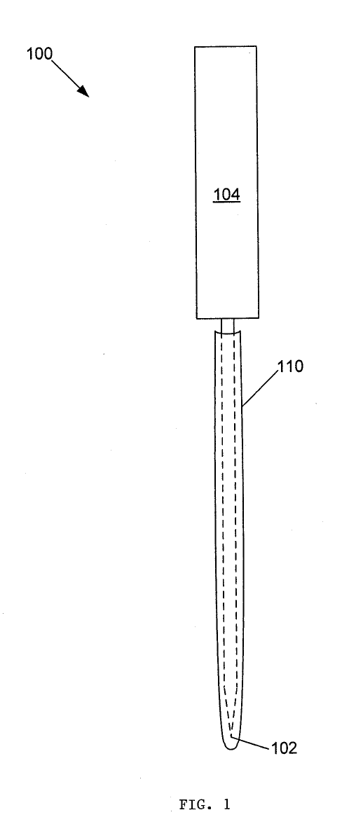

Referring to FIG. 1, tissue-piercing member is shown as an acupuncture needle (100) comprising a coating (110). Here the acupuncture needle (100) is a solid, elongate rod, but in other variations, it may have one or more lumens. The acupuncture needle (100) may be made from any suitable material, e.g., a metallic material such as stainless steel and alloys thereof. In other variations, the acupuncture needle (100) may be made from plastic or silica. The acupuncture needle (100) may be polished or coated with gold or silver.

The acupuncture needle (100) may have a sharp distal end (102) for insertion into skin at an acupuncture point. The acupuncture needle (100) may also have various lengths and/or diameters. For example, between about a 25 gauge to about a 40 gauge acupuncture needle may be used. In some variations, use of a 31 gauge acupuncture needle may be beneficial. The distal end (102) may be inserted to any depth appropriate for acupuncture therapy. For example, the distal end (102) may be inserted from about 2 mm to about 12 mm, about 4 mm to about 12 mm, about 6 mm to about 12 mm, about 8 mm to about 12 mm, or about 8 mm to about 12 mm under the skin. The distal end (102) may, in some variations, be blunted, polished, or modified by a practitioner prior to use.

The proximal end of the acupuncture needle (100) may include an optional handle (104). The handle (104) may provide greater control and/or facilitate insertion and/or removal of the acupuncture needle (100) from the skin. The handle (104) may be made of any suitable material and may be of any suitable size, shape, or length. For example, the handle (104) may be from about 2 cm to about 32 cm in length. In FIG. 1 the handle (104) is shown as being aligned with the shaft of the acupuncture needle (100), but other handle configurations are also contemplated. For example, the longitudinal axis of the handle (104) may form an angle of about 300, about 40°, about 50°, about 60°, about 70°, about 800, or about 90° with the longitudinal axis of the acupuncture needle (100). In some variations, the handle (104) comprises coiled copper. In other variations, the handle (104) or a portion thereof, is made of stainless steel or plastic.

At least a portion of the shaft of the acupuncture needle (100) may comprise a coating (110) that includes an agent. The agent may be provided as a solid, a liquid, a semi-solid, etc., which may be applied using know techniques such as spray coating or dip coating, to coat the acupuncture needle (100). The coating (110) may be permanent or semi-permanent, and may be applied to the entire acupuncture needle or a portion thereof. In some variations, the coating (110) is uniformly applied to the shaft of the needle. In other variations, the coating (110) may be thicker in some areas of the acupuncture needle (100). Thicker areas of the coating may contain higher concentrations of the agent. In some variations it may be beneficial to have a higher concentration of an agent at the distal end (102) of the acupuncture needle (100). The agent and the type of coating used may be selected based on the specific tissue-piercing member being used, the medical condition of the patient, and the desired duration of therapy.

In some variations, the coating may be applied immediately prior to insertion, e.g., by dipping the distal end of a tissue-piercing member into the agent. In other variations, the agent may be spread along the entire shaft of the tissue-piercing member prior to insertion into the skin. In further variations, the tissue-piercing member or a portion thereof, e.g., the distal end of the tissue-piercing member, may be passed through the agent. In this instance, the agent may be topically applied to an area of the skin at an acupuncture point or an area adjacent thereto, prior to insertion of the tissue-piercing member into either area.

In use, the coating on the tissue-piercing member may be deposited, dissolved, absorbed, or otherwise delivered to the surrounding tissue. The coating may accentuate the effects of acupuncture therapy, minimize pain or inflammation caused by acupuncture therapy, or increase the effectiveness or duration of the effectiveness of the acupuncture therapy. In some variations, because the coating may affect a larger area of tissue than just the acupuncture needle, the tissue-piercing member may be inserted adjacent to the acupuncture point to treat a medical condition.

In some variations, the proximal end of the tissue-piercing member, e.g., an acupuncture needle, is configured to include a reservoir. The reservoir may be a frangible reservoir containing one or more agents. For example, the frangible reservoir may be configured to include an inner vial that may be broken by bending an outer casing. In this instance, breaking the vial may cause an agent housed within the inner vial to mix with another agent. Further, the frangible reservoir may include an outlet through which the agent or a mixture of agents can flow. In other variations, the outlet may be sealed to allow the agents to mix. The seal may be broken by squeezing, twisting, pushing, or by manipulation of the handle. In one exemplary method of using the frangible reservoir, a tissue-piercing member is inserted into skin at an acupuncture point or an area adjacent thereto, to a desired depth. The user bends the handle while keeping the tissue-piercing member stationary to cause an inner vial containing an agent to break. The agent may then flow through an outlet in the reservoir and through either a lumen within the tissue-piercing member that is in fluid communication with the outlet, or along the exterior surface of the tissue-piercing member towards or into the skin.

As previously stated, the tissue-piercing members may be coated or impregnated with an agent. Thus, acupuncture needle (100) may be coated upon insertion into the skin by first passing the distal end (102) through a layer of the agent applied topically to the skin at or adjacent to the acupuncture point. As the distal end (102) of the acupuncture needle (100) is inserted, a small amount of the agent may adhere to the shaft which may then be delivered to the surrounding tissue. Methods of impregnating a device with an agent are generally known to those skilled in the art. For example, the acupuncture needle (100) may include a hollow portion containing the agent. Alternatively or additionally, the acupuncture needle (100) may include a plurality of pores that retain the agent until the acupuncture needle is inserted into tissue.

In some variations, the tissue-piercing member is configured as a single lumen needle (not shown). The single lumen needle may have a distal end for penetrating skin and a proximal end connectable to a reservoir. The single lumen needle may be any suitable length. Generally, the single lumen needle has a length of less than about three centimeters. The single lumen needle may also have any suitable gauge. For example, the needle may be a small gauge needle, e.g., a 28 gauge to a 40 gauge needle. Any suitable type of reservoir may also be used, including, e.g., a syringe, a cartridge, a vial, or a frangible vial. The reservoir may contain an agent. For example, the agent may be lidocaine or epinephrine.

To treat a medical condition, the single lumen needle may be attached to the reservoir. The needle may be permanently attached or removably attached. The needle may be inserted into skin at or adjacent to an acupuncture point to a desired depth. For example, the needle may be inserted to a depth of about 5 mm. The contents of the reservoir comprising the agent may then be delivered to the acupuncture point via the single lumen needle. For example, the contents may be delivered by pressing the plunger of a syringe.

In other variations, the tissue-piercing member is configured as an injection pen. An injection pen may be a small gauge (e.g., 28 to 40 gauge) needle connected to a cartridge within a housing that is pre-filled with one or more agents. The cartridge may be a blister located adjacent to a distal end of the small gauge needle. The needle of the injection pen may be set to a penetration depth between about 2 mm and about 7 mm by a user. To inject a fluid, the distal end of the needle may pierce the blister before advancing into the tissue, e.g., skin. This delivers the fluid from the blister into the tissue. The blister pack may be rigid or flexible. The injection pen may also be programmable by a user to deliver a specified amount of an agent by, for example, adjusting a dial. Examples of injection pens are generally known and include insulin pens, e.g., Humalog® pen (Eli Lilly and Company), Byetta® pen (Amylin-Lilly), and the OptiClick® pen (Lantis). The agent may include, for example, bupivicaine.

In some variations, the tissue-piercing member is configured as a single lumen needle connected to a syringe pump. Here the agent may be stored in a reservoir connected to the syringe pump. Any suitable reservoir, as previously described, may be used. The syringe pump may be programmable. For example, the syringe pump may operate to deliver the agent to the acupuncture point on a continuous, periodic, or repeat basis.

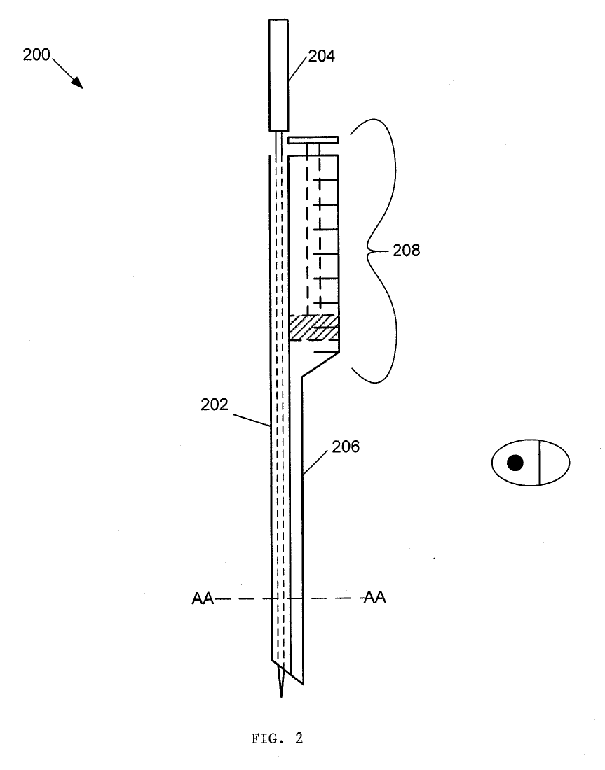

Some devices may be configured to include two or more tissue piercing members, as shown in FIG. 2. In this figure, dual lumen needle (200) may be used in combination with an acupuncture needle (204). The dual lumen needle (200) includes side-by-side lumens. A first lumen (202) may be configured to allow the acupuncture needle (204) to pass therethrough and a second lumen (206) may be connected to a syringe (208) containing an agent at the proximal end of the dual lumen needle (200). The syringe (208) may be permanently or removably attached to the dual lumen needle (200). The syringe may contain any suitable amount of an agent. In other variations not shown, the second lumen (206) may be connected to a cartridge, a vial, or a frangible vial. In some variations, the acupuncture needle (204) is the dual lumen needle. The first and second lumens may have the same diameter or different diameters. For example, the first lumen (202) may have a larger or smaller diameter than the second lumen (206).

The dual lumen needle (200) may be used at any acupuncture point or area adjacent thereto to relieve pain or treat a medical condition. For example, the acupuncture needle (204) may be inserted in the skin before, simultaneously to, or after the dual lumen needle (200). In some variations, the acupuncture needle (204) may be allowed to remain in the skin after the dual lumen needle (200) is removed. In other variations, the acupuncture needle (204) may lack a handle so that the acupuncture needle (204) is able to pass completely through the first lumen (202). When a syringe (208) is employed, a user may inject a portion of the agent contained therein. The user may then remove the syringe while leaving the acupuncture needle (204) in place. This procedure, or a variation thereof, may then be repeated using another traditional acupuncture needle (204) at another location on the individual's body.

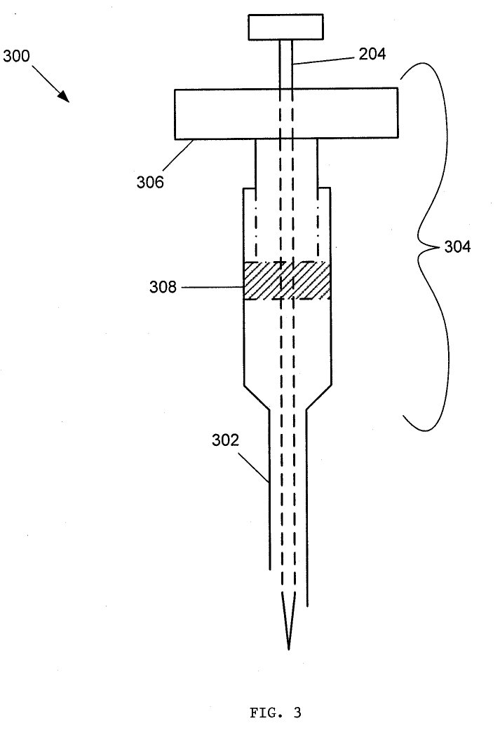

Another variation of the devices described here is illustrated in FIG. 3. In this figure, the device comprises an injection device (300) and an acupuncture needle (204). The injection device (300) may include a tissue-piercing member (302), a syringe (304), and a passage for an acupuncture needle (204). The passage may extend from the plunger (306) of the syringe (304) through the septum (308) and to the distal end of the tissue-piercing member (302). In other variations, the passage may extend partially through the injection device. For example, the injection device may include a side lumen through which a malleable or bendable acupuncture needle is passed. The side lumen may allow the acupuncture needle to be inserted without first passing through one or more membranes, e.g., the septum (308). This may reduce wear on the distal end of the acupuncture needle. The tissue-piercing member (302) may comprise a needle, a cannula, or the like. The tissue-piercing member (302) may have an internal diameter large enough to accommodate passage of an acupuncture needle (204) to the distal end. In some variations, the internal diameter of the tissue-piercing member (302) is large enough to accommodate passage of the acupuncture needle (204) and an agent contained in the syringe (304).

The syringe (304) will generally be configured to contain an agent. The agent may include bupivicaine, for example. A user may inject the agent through the distal end of the tissue-piercing member (302). To allow an acupuncture needle (204) to pass therethrough, the syringe (304) may include a passage or area that includes a pierceable region (not shown), e.g., through the center of the plunger (306) and/or the septum (308). In some variations, the pierceable region may be configured to be self-sealing after being punctured by an acupuncture needle. The acupuncture needle (204) may pass through the agent contained within the syringe (304) before insertion into the surface of the skin.

According to some modes of use, the injection device (300) may be used to insert a plurality of traditional acupuncture needles into an individual. To illustrate, the user may begin by locating a desired acupuncture point and injecting a small amount of an agent therein. The user may then advance an acupuncture needle (204) through the injection device (300) as described above. After the acupuncture needle (204) is in place, the user may withdraw the injection device (300) from the acupuncture point, leaving the acupuncture needle (204). Upon withdrawal, the user may then locate another desired acupuncture point and repeat the above process to insert another traditional acupuncture needle.

The injection device (300) is provided as an illustrative example. Other injection devices will be apparent to those skilled in the art and may include, but are not limited to, auto-injectors, side-lumen needles, and/or intravenous devices such as needle and syringe devices, line devices, ports, and catheter devices. In other variations, the syringe on an injection device can be attached or joined to electrodes, a TENS unit, LEDs or lights, ultrasound probes, detachable magnets, very small gauge needles or microneedles, magnetized needles, a heat source, a negative pressure vacuum, or combinations thereof.

### Other Devices

Other devices that may be used to deliver the agent alone or in combination with a tissue-piercing member include an implantable reservoir, an external reservoir, or a patch. The implantable reservoirs may include a marker, such as an ultrasonic marker, that can be detected by imaging technology. In some variations, the implantable devices may be refillable or replaceable once implanted. When patches are used, the patch may include a plurality or array of tissue piercing members. These devices may be used to treat chronic medical conditions where longer term therapy is required.

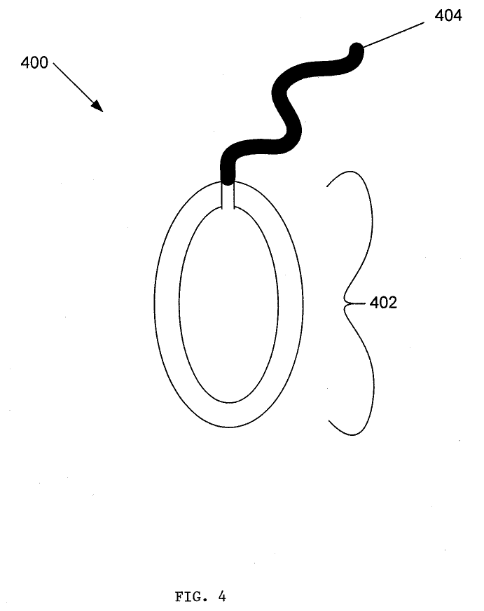

An implantable reservoir containing the agent may be positioned beneath the skin at or near an acupuncture point. As shown in FIG. 4, an implantable reservoir (400) may include a reservoir (402) and an outlet connected to a wick (404). The implantable reservoir (400) may be subcutaneously implanted at or adjacent to an acupuncture point. The implantable reservoir (400) may be used, for example, to treat a chronic condition such as chronic joint pain due to osteoarthritis.

The reservoir (402) may contain an agent. The reservoir (402) may be fabricated using a biocompatible and/or biodegradable material such as silastic, a lipophilic polymer matrix, or hydrophilic polymer matrix. The reservoir (402) may include capsules, a liquid-type dispersing medium, or a solid-type dispersing medium. The reservoir (402) may be of any suitable size or shape. The agent may be released from the reservoir (402) by diffusion, by dissolution, by erosion of polymers, by swelling of polymers, by activation, or by magnetism.

In some variations, the reservoir (402) is configured to optionally include an outlet connected to a wick (404). The wick (404) may be capable of delivering the agent to an acupuncture point or area adjacent thereto. In one variation, the wick (404) may be selected based on a rate of delivery to the acupuncture point. The wick (404) may be of any suitable length. For example, the wick (404) and may range from nanometers to micrometers to centimeters in length. In some variations, the length of wick (404) may range from about 0.5 cm to about 10 cm in length. By delivering a fairly continual amount of the agent to an acupuncture point, the wick may act as a longer term acupuncture therapy for medical conditions.

The internal reservoirs may also comprise components such as electrodes, magnets (that can be influenced by cutaneous application of an electric current on the skin surface and vice versa), electromagnets, crystals, piezoelectric crystals which can convert mechanical strain applied externally into electric current, needles, stents, ports, microspheres, seeds, nanoparticles, nanopowders, nanocrystals, quantum dots, colloidal gold, colloidal silver, iron nanoparticles, and fullerenes, biologics such as vaccines, blood and blood components, allergenics, somatic cells, tissues, cells, sugars, proteins, nucleic acids, antibodies, or a combination of these substances, liquid nitrogen, thermoelectric and thermoconductive materials that can absorb heat or current applied externally from a heat or current-generating source, light and/or heat emitting devices such as LEDs, conductive electrode gels, oxides, ice, frozen gels, pacemakers, microsensors, or combinations thereof.

In other variations, the devices are external reservoirs. The external reservoirs may include a bladder that is flexible and configured to store the agent. The external reservoirs may include one or more agents. In some variations, the external reservoirs may be packaged as a single dose. Multiple doses may also be packaged. Blisters, wipes, or other impregnated materials may also be used as external reservoirs.

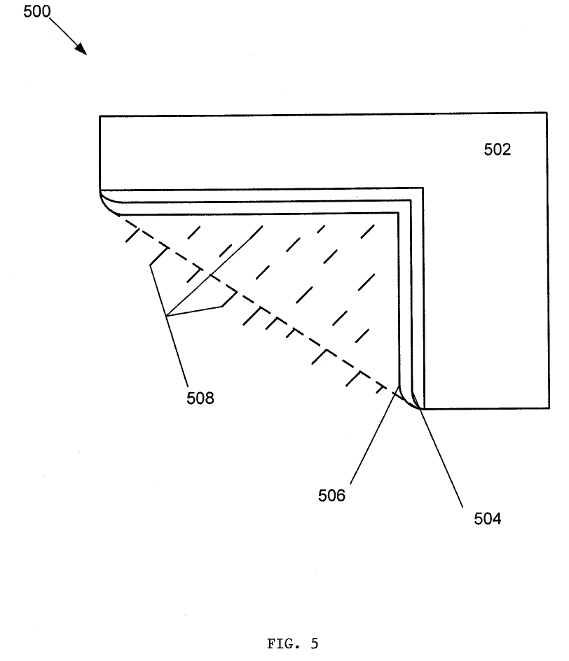

Some variations of the external reservoirs are configured as skin patches. Referring to FIG. 5, patch (500) may include a top layer (502), an optional middle layer or reservoir (504), and an adhesive layer (506). The patch (500) may be of any suitable size or shape. The patch (500) may be adhered to the surface of skin at an acupuncture point for providing a continuous, or nearly continuous, dose of an agent over an extended period of time. For example, the agent may include buticaine. In some variations, the extended period of time may be one hour, one day, two days, three days, or one week.

The patch (500) may generally include an agent, in a layer (504), or “reservoir,” underlying an upper backing layer, such as top layer (502). The laminated structure may contain a single reservoir, or it may contain multiple reservoirs. When multiple reservoirs are employed, they may include the same agent or different agents, or each reservoir may include a combination of agents. The patch (500) may also be configured to include a component that modifies delivery of an agent therefrom. For example, a rate-limiting membrane may be placed between the reservoirs to modify release of the agent.

In some variations, the reservoirs may comprise a polymeric matrix of a pharmaceutically acceptable adhesive material that serves to affix the patch to the skin. For example, the adhesive material may be a pressure-sensitive adhesive (PSA) including, but not limited to, polyethylenes; polysiloxanes; polyisobutylenes; polyacrylates; polyacrylamides; polyurethanes; plasticized ethylene-vinyl acetate copolymers; and tacky rubbers such as polyisobutene, polybutadiene, polystyrene-isoprene copolymers, polystyrene-butadiene copolymers, and neoprene (polychloroprene).

The backing layer (502) may function as the primary structural element of the patch and may provide the patch (500) with flexibility and in certain variations, occlusivity. The material used for the backing layer (502) is generally inert and incapable of absorbing the agent contained within the reservoirs of the patch. The backing layer (502) may be comprised of a flexible elastomeric material that serves as a protective covering to prevent loss of agent and/or carrier via transmission through the upper surface of the patch (500), and may impart a degree of occlusivity to the patch (500), such that the area of the body surface covered by the patch (500) becomes hydrated during use. The material used for the backing layer (502) may permit the patch (500) to follow the contours of the skin and be worn comfortably on areas of skin such as at joints or other points of flexure that are normally subjected to mechanical strain, with little or no likelihood of the patch (500) disengaging from the skin due to differences in the flexibility or resiliency of the skin and the patch (500). The materials used as the backing layer may be either occlusive or permeable, as noted above, and may be made from synthetic polymers (e.g., polyester, polyethylene, polypropylene, polyurethane, polyvinyl chloride, and polyether amide), natural polymers (e.g., cellulosic materials), or macroporous woven and nonwoven materials.

During storage and prior to use, the laminated structure may include a release liner (not shown). Immediately prior to use, this liner is typically removed from the device so that the patch (500) may be affixed to the skin. The release liner may be made from an agent/carrier impermeable material, and may be prepared as a disposable element that serves only to protect the patch (500) prior to application. The release liner may be formed from a material impermeable to the agent, and which is easily stripped from the patch prior to use.

In another variation, the agent-containing reservoir and skin contact adhesive (506) are present as separate and distinct layers, with the adhesive (506) underlying the reservoir. In such a case, the reservoir may be a polymeric matrix as described above. Alternatively, the reservoir may be comprised of a liquid or semisolid formulation contained in a closed compartment or “pouch,” or it may be a hydrogel reservoir, or it may take some other form. Hydrogels are generally macromolecular networks that absorb water and thus swell, but may or may not dissolve in water. That is, hydrogels contain hydrophilic functional groups that provide for water absorption, but the hydrogels are comprised of crosslinked polymers that may give rise to aqueous insolubility. Generally, then, hydrogels are comprised of crosslinked hydrophilic polymers such as a polyurethane, a polyvinyl alcohol, a polyacrylic acid, a polyoxyethylene, a polyvinylpyrrolidone, a poly(hydroxyethyl methacrylate) (poly(HEMA)), or a copolymer or mixture thereof.

Additional layers, e.g., intermediate fabric layers and/or rate-controlling membranes, may also be present in any of the patches (500). Fabric layers may be used to facilitate fabrication of the patch (500), while a rate-controlling membrane may be used to control the rate at which an agent permeates out of the patch (500). A rate-controlling membrane, if present, may be included in the patch (500) on the skin side of one or more of the agent reservoirs. The materials used to form such a membrane may be selected to limit the flux of one or more agents contained in the patch (500). Representative materials useful for forming rate-controlling membranes include, but are not limited to, polyolefins such as polyethylene and polypropylene, polyamides, polyesters, ethylene-ethacrylate copolymer, ethylene-vinyl acetate copolymer, ethylene-vinyl methylacetate copolymer, ethylene-vinyl ethylacetate copolymer, ethylene-vinyl propylacetate copolymer, polyisoprene, polyacrylonitrile, ethylene-propylene copolymer, and the like.

The patches (500) may be fabricated using conventional coating and laminating techniques known in the art. For example, adhesive matrix systems can be prepared by casting a fluid admixture of adhesive, active agent, and carrier onto the backing layer (502), followed by lamination of the release liner (not shown). Similarly, the adhesive mixture may be cast onto the release liner, followed by lamination of the backing layer (502). Alternatively, the agent reservoir may be prepared in the absence of agent or excipient, and then loaded by “soaking” in an agent/carrier mixture. In general, these patches are fabricated by solvent evaporation, film casting, melt extrusion, thin film lamination, die cutting, or the like.

In certain variations, an adhesive overlayer that also serves as a backing for the patch (500) may be used to better secure the patch (500) to the body surface. This overlayer may be sized such that it extends beyond the agent reservoir so that adhesive on the overlayer comes into contact with the body surface. The overlayer may be useful because the adhesive/agent reservoir layer may lose its adhesion a few hours after application due to hydration. By incorporating such an adhesive overlayer, the patch may remain in place for the required period of time.

The adhesive layer (506) may additionally include at least one microprotrusion (508) (as a tissue-piercing member). The at least one microprotrusion (508) is configured to pierce into the stratum corneum of the skin. By piercing only the top surface of the skin, the microprotrusions (508) more effectively deliver the agent to an acupuncture point than topical application. The microprotrusions (508) may be 1.0 mm or less in length. In some variations, the microprotrusions (508) may be uniform and/or uniformly distributed over the adhesive layer (506).

In operation, the patch (500) may be applied to the skin of an individual at, or adjacent to, an acupuncture point. The microprotrusions (508) may pierce the stratum corneum. An agent stored between the top layer (502) and the adhesive layer (506) may flow through the adhesive layer (506) to the microprotrusions (508). At least a portion of the agent may flow down the at least one microprotrusion to a layer of the epidermis. Another portion of the agent may remain on the surface of the skin.

Selection of the device to be employed will generally depend on such factors as the medical condition being treated, patient tolerance, and interactions with concurrent medical therapies. The configuration of the tissue-piercing member and the agent used may also depend on the particular tissue-piercing member selected and the age and general health of the individual being treated.

### II. AGENTS

The acupuncture and acupressure therapies described herein generally include the delivery of one or more agents. The agents may be provided in any suitable form or in any suitable formulation. For example, the agents may be provided as liquids, solids, semi-solids, or combinations thereof. The agents may also be provided in any suitable dosage form, including, but not limited to, topical dosage forms, injectable dosage forms, and intravenous dosage forms. In some variations, the dosage forms, or portions thereof, may be formulated for immediate release, controlled release, delayed release, extended release, or timed release.

Agents

The agents described herein may be beneficial in relieving pain or treating a medical condition. For example, the agent may be selected to treat allergic disorders, cardiovascular disorders, gastrointestinal motility disorders, hormonal disorders, immune disorders, nervous disorders, respiratory disorders, skeletal disorders, urogenital disorders, any combination of the foregoing, and the like. In general, the agent may extend the amount of time that the acupuncture therapy is effective and/or correct for an inaccuracy in placing the acupuncture needle. It is understood that the terms “agent,” “active agent,” and “drug” are used interchangeably herein throughout.

The agents that may used with the devices described here include without limitation, anti-atherosclerotic agents, anti-psoriatics, antispasmodics, muscle relaxants, muscle contractants, histamines, antipyretics, analgesics, antihypertensives, anticoagulants, procoagulants, cholesterol-reducing agents, anticonvulsants, cognitive enhancers, cholinergics, anti-cholinergics, anti-Alzheimer's disease agents, sedatives, anti-Parkinson substances, hypnotics, anti-psychotic substances, antacids, antihistamines, antidiabetics, contraceptives, sympathomimetics, coenzymes, adrenergics, adrenergic antagonists, enzyme inhibitors, neurotoxins, neurotransmitters, hormones, anti-ulcer agents, antiflatulents, proton pump inhibitors, antidiarrheals, antipruritics, anti-emetics, antireflux agents, antiobesity agents, autoimmune disorder agents, anti-cancer substances, immunomodulatory factors, anti-glaucoma compounds, anti-inflammatory agents, anti-vertigo medications, local anesthetics, diuretics, ophthalmics, trophic factors, growth factors, nucleic acids, anti-infectives, vitamins, minerals, nutritional supplements, lubricants, imaging agents, emulsifying stabilizers, herbs, plant extracts, astringents, combinations, derivatives, or precursors thereof.

For example, muscle relaxants include, but are not limited to, laxatives, tranquilizers, and tranquilizers. Exemplary sympathomimetics include epinephrine, norepinephrine, and dopamine. Exemplary antihypertensives include nitrates. Examples of cholesterol reducing agents include cholesterol ester transfer protein inhibitors. Examples of anti-psychotics include anti-depressants and serotonin. Examples of hypnotics include enkephalin and opioids. Examples of histamines include H2-blocking agents. Examples of anti-inflammatories include steroids and corticosteroids. Examples of anti-infectives include antifungals, antibiotics, anti-viral substances, vaccines, antiseptics, anti-parasite compounds, anti-protozoal compounds, anti-AIDS substances, and cough or cold remedies. Examples of vitamins include tocopheryl and retinol. Examples of minerals include niacin, iron and ferric sulfate. Examples of nutritional supplements include hyaluronic acid, nutraceuticals, phenol, polyphenols, isoflavones, resveratrol, soy isoflavones, grape seed extract, polyphenols, curcumin, and epigenin. Examples of lubricants include pharmaceutical grade oils, oily solvents, fatty acids and fatty acid esters, moisturizers, silicone, silicone rubber, rubber, latex, alcohol, and saline. Examples of plant extracts include anti-inflammatory plant extracts, aloe vera extract, Echinacea extract, chamomile hammamelis extract, Chinese zizipus jujuba, feverfew parthenolides, and carotenoids such as beta-carotene, lycopene, astaxanthons, and lutein. Examples of enzymes include prostaglandins, glycogen phosphorylase inhibitors, and phospholipids. An example of an anti-ulcer agent is sucralfate. Examples of anti-emetics include anti-nauseants and anti-motion sickness medications. An example of a growth factor is endothelial growth factor. An example of a neurotoxin includes botulinum toxin type A. Exemplary ophthalmics include miotics. An example of a immunomodulator factors is an immunosuppressant.

In some variations, it may be beneficial to use a local anesthetic as the agent. Here the agent may comprise a local anesthetic such as lidocaine, high dose lidocaine, xylocaine, bupivicane, buticaine, epinephrine, combinations, derivatives, or precursors thereof. For example, the local anesthetic may be combined with epinephrine and/or a corticosteroid. In other variations, lidocaine with epinephrine with dilution ranging from 1:1 to 1:200000 is used. Other agents that may be employed alone or in combination with the local anesthetic include triamcinolone, saline, capryllic/capric triglycerides, combinations, derivatives, or precursors thereof.

Selection of the agent to include in the agent may depend on the acupuncture therapy administered, medical condition of the individual, and severity or refactoriness of potential side-effects. For example, acupuncture therapy may relieve pain for only a short period of time. In view of this, a long-lasting agent may be employed in combination with the acupuncture therapy.

Dosage Forms

The agents described here may be formulated into any dosage form, including, but not limited to, topical dosage forms, injectable dosage forms, and intravenous dosage forms. The dosage forms may also be adapted for any type of drug release, e.g., immediate release, controlled release, delayed release, extended release, or timed release. Other ingredients, such as pH buffering agents, binders, disintegrants, diluents, emulsifying agents, fillers, lubricants, penetration enhancers, wetting agents, flavoring agents, colorants, and preservatives, may also be included in the dosage forms.

Selection of the dosage form to administer may depend on such factors as the particular device and/or agent being delivered and the medical condition being treated. A more detailed description of some of these dosage forms is provided below.

1) Topical Dosage Forms

The agents described herein may be formulated into any topical dosage form. The topical dosage forms may be creams, lotions, solutions, gels, ointments, pastes, patches, etc. The topical dosage forms generally include an agent, and are suitable for application to any body surface, including mucosal body surfaces.

Various additives may also be included in the topical dosage forms. For example, solvents, including relatively small amounts of alcohol, may be used to solubilize certain formulation components. Penetration enhancers may also be included. Examples of suitable penetration enhancers include, but are not limited to, ethers such as diethylene glycol monoethyl ether; diethylene glycol monomethyl ether; surfactants such as sodium laurate, sodium lauryl sulfate, cetyltrimethylammonium bromide, benzalkonium chloride, Poloxamer (231, 182, 184), Tween (20, 40, 60, 80), and lecithin; alcohols such as ethanol, propanol, octanol, benzyl alcohol, and the like; polyethylene glycol and esters thereof, such as polyethylene glycol monolaurate; amides and other nitrogenous compounds such as urea, dimethylacetamide (DMA), dimethylformamide (DMF), 2-pyrrolidone, 1-methyl-2-pyrrolidone, ethanolamine, diethanolamine, and triethanolamine; terpenes; alkanones; and organic acids; and sulfoxides such as DMSO.

In some variations, the topical dosage form is an ointment. The ointment base may be an oleaginous base, an emulsifiable base, an emulsion base, or a water-soluble base. The oleaginous ointment base that may be used includes, without limitation, vegetable oils, fats obtained from animals, and semisolid hydrocarbons obtained from petroleum. Suitable emulsifiable ointment bases that may be used, include, for example, hydroxystearin sulfate, anhydrous lanolin, and hydrophilic petrolatum. Exemplary emulsion ointment bases that may be used are water-in-oil (W/O) emulsions or oil-in-water (O/W) emulsions that include, for example, cetyl alcohol, glyceryl monostearate, lanolin, and stearic acid.

In other variations, the topical dosage form is a cream. The creams may be viscous liquids or semisolid emulsions, either oil-in-water or water-in-oil. The cream bases may be water-washable, and contain an oil phase, an emulsifier, and an aqueous phase. The oil phase, or internal phase, may be generally comprised of petrolatum and a fatty alcohol such as cetyl or stearyl alcohol. The aqueous phase may be formulated to exceed the oil phase in volume, and contain a humectant.

In yet another variation, the topical dosage form is a gel. The gels may be semisolid, suspension-type systems. Single-phase gels may contain organic macromolecules distributed substantially uniformly throughout the carrier liquid, which may be aqueous, but may also contain an alcohol and, optionally, an oil. Exemplary organic macromolecules that may be used in the gels, include, but are not limited to, carbomers; hydrophilic polymers such as polyethylene oxides, polyoxyethylene-polyoxypropylene copolymers, and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin.

In yet further variations, the topical dosage form is a lotion. The lotions may be formulated as suspensions of solids and contain suspending agents to produce better dispersions. Examples of such suspending agents include methylcellulose and sodium carboxymethylcellulose.

The topical dosage forms may also be formulated as a paste. Pastes are semisolid dosage forms in which the active agent is suspended in a suitable base. Depending on the nature of the base, pastes are divided between fatty pastes or those made from single-phase aqueous gels. The base in a fatty paste is generally petrolatum, hydrophilic petrolatum, or the like. The pastes made from single-phase aqueous gels may generally incorporate carboxymethylcellulose or the like as a base.

In some variations, the topical dosage forms are prepared with liposomes, micelles, or microspheres. Liposomes are microscopic vesicles having a lipid wall comprising a lipid bilayer. Liposome formulations may be used for poorly soluble or insoluble drugs. Liposomal preparations for use in the dosage forms described here include cationic (positively charged), anionic (negatively charged), and neutral preparations. Cationic liposomes are readily available. For example, N[1-2,3-dioleyloxy)propyl]-N,N,N-triethylammonium (DOTMA) liposomes are available under the trade name Lipofectin® (GIBCO BRL, Grand Island, N.Y.). Anionic and neutral liposomes are readily available as well, e.g., from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), and dioleoylphoshatidyl ethanolamine (DOPE), among others. These materials can also be mixed with DOTMA in appropriate ratios. Methods for making liposomes using these materials are well known.

Micelles are comprised of surfactant molecules arranged so that their polar head groups form an outer spherical shell, while their hydrophobic, hydrocarbon chains are oriented towards the center of the sphere, forming a core. Micelles form in an aqueous solution containing surfactant at a high enough concentration so that micelles naturally result. Surfactants useful for forming micelles include, but are not limited to, potassium laurate, sodium octane sulfonate, sodium decane sulfonate, sodium dodecane sulfonate, sodium lauryl sulfate, docusate sodium, decyltrimethylammonium bromide, dodecyltrimethylammonium bromide, tetradecyltrimethylammonium bromide, tetradecyltrimethylammonium chloride, dodecylammonium chloride, polyoxyl 8 dodecyl ether, polyoxyl 12 dodecyl ether, nonoxynol 10, and nonoxynol 30. Micelle formulations for use in the topical dosage forms herein described can be either incorporated into the reservoir of a topical device, e.g., a patch, or into a formulation to be applied to the body surface.

Similarly, microspheres may be incorporated into the topical dosage forms. Like liposomes and micelles, microspheres essentially encapsulate a drug or drug-containing formulation. Microspheres are generally, although not necessarily, formed from synthetic or naturally occurring biocompatible polymers, but may also be comprised of charged lipids such as phospholipids. Preparation of microspheres is well known and described in pertinent texts and literature.

Various additives may also be included in the topical dosage forms. For example, solvents, including relatively small amounts of alcohol, may be used to solubilize certain dosage form components. Penetration enhancers may be added. Examples of suitable penetration enhancers include, but are not limited to, ethers such as diethylene glycol monoethyl ether (available commercially as Transcutol®) and diethylene glycol monomethyl ether; surfactants such as sodium laurate, sodium lauryl sulfate, cetyltrimethylammonium bromide, benzalkonium chloride, Poloxamer (231, 182, 184), Tween (20, 40, 60, 80), and lecithin; alcohols such as ethanol, propanol, octanol, benzyl alcohol, and the like; polyethylene glycol and esters thereof such as polyethylene glycol monolaurate (PEGML); amides and other nitrogenous compounds such as urea, dimethylacetamide (DMA), dimethylformamide (DMF), 2-pyrrolidone, 1-methyl-2-pyrrolidone, ethanolamine, diethanolamine, and triethanolamine; terpenes; alkanones; and organic acids such as citric acid and succinic acid. Azone® and sulfoxides such as DMSO and C₁₀ MSO may also be used.

The topical dosage forms may also include conventional additives such as opacifiers, antioxidants, fragrance, colorants, gelling agents, thickening agents, stabilizers, surfactants, and the like. Other agents may also be added, such as antimicrobial agents, to prevent spoilage upon storage, i.e., to inhibit growth of microbes such as yeasts and molds. Suitable antimicrobial agents are typically selected from the group consisting of the methyl and propyl esters of p-hydroxybenzoic acid (i.e., methyl and propyl paraben), sodium benzoate, sorbic acid, imidurea, and combinations thereof.

The dosage forms may also contain irritation-mitigating additives to minimize or eliminate the possibility of skin irritation resulting from the agent. Suitable irritation-mitigating additives include, for example, alpha.-tocopherol; monoamine oxidase inhibitors, e.g., phenyl alcohols such as 2-phenyl-1-ethanol; glycerin; salicylic acids and salicylates; ascorbic acids and ascorbates; ionophores such as monensin; amphiphilic amines; ammonium chloride; N-acetylcysteine; cis-urocanic acid; capsaicin; and chloroquine.

2) Other Dosage Forms

The agent may also be formulated into other parental dosage forms. The drugs may be formulated for administration by injection, e.g., by bolus injection or infusion (continuous or intermittent). Such dosage forms may be prepared by dissolving, suspending, or emulsifying the drugs in an aqueous or nonaqueous solvent, such as vegetable or other similar oils, synthetic aliphatic acid glycerides, esters of higher aliphatic acids or propylene glycol, and if desired, with conventional additives such as solubilizers, isotonic agents, suspending agents, emulsifying agents, stabilizers and preservatives.

In one variation, the injectable dosage form is prepared as an aqueous solution, using Hanks's solution, Ringer's solution, or normal saline. Formulations for injection may be presented in unit dose form, e.g., in ampules or in multi-dose containers within a cartridge, reservoir, etc., with an added preservative. The agents may take such forms as suspensions, solutions or emulsions in oily or aqueous vehicles, and may contain formulatory agents such as suspending, stabilizing and/or dispersing agents.

The injectable dosage form may further be prepared as an oily suspension of drug. Suitable lipophilic solvents or vehicles for use in this instance include fatty oils such as sesame oil, or synthetic fatty acid esters, such as ethyl oleate or triglycerides, or liposomes. Aqueous injection suspensions may contain agents which increase the viscosity of the suspension, such as sodium carboxymethyl cellulose, sorbitol, or dextran. Optionally, the suspension may also contain suitable stabilizers or agents which increase the solubility of the drugs to allow for the preparation of highly concentrated solutions. Alternatively, the drugs may be in powder form for constitution with a suitable vehicle, e.g., sterile water, normal saline, etc., before use.

### III. METHODS

1) Administration

The agents described here may be administered in any suitable manner using the devices disclosed herein. For example, the agents may be administered via topical (including transdermal), intravenous, and subcutaneous routes.

As previously mentioned, the agent that treats a medical condition or relieves pain may be administered in combination with acupuncture therapy. In some variations, combined administration occurs as a result of use of a coated needle, a dual lumen needle, an injection device, an implantable device, a patch, or passing an acupuncture needle through a topically applied agent. Administration of the agent and the acupuncture therapy may be repeated as often as desired.

The agents may be administered directly into or in the vicinity of an acupuncture or acupressure point. The agents can be administered alone or in combination with other agents. The agents may also be administered via transdermal delivery, as fluids, alone or in combination with heat and sound over acupuncture/pressure points through electrophoresis, iontophoresis and phonophoresis to induce a synergistic effect. Electromagnetophoresis with electromagnetic signals administered simultaneously in conjunction with drug delivery is also contemplated. The effect of iontophoresis or electrophoresis, for example, could be potentiated if combined with pretreatment with other physical enhancement methods such as pulsing of high voltages (electroporation), low frequency ultrasound, and laser to enhance the transdermal delivery of agents. A laser can also be used for enhancing transdermal absorption of drugs across acupuncture/acupressure points due to stratum corneum removal.

2) Dosing Regimens

The dosing regimen employed may depend on a number of factors, such as the medical condition being treated, severity of symptoms or pain, and the responsiveness of the medical condition or pain to the agent. The dosing regimen may provide one or more doses per day of the agent, and may continue for several hours, for one day to several days, or for several months or more. In general, the dosing regimen will continue until the underlying medical condition is alleviated or until the pain is relieved.

The agent may be provided in any suitable amount. For example, when the agent is provided in a formulation, it may be included in amounts of about 1% to about 99% by weight of the formulation. In one variation, the agent is included in the formulation in an amount of about 1% to about 30% by weight of the formulation. It is understood that the above listed amounts are exemplary, and that there may be instances in which higher or lower amounts may be merited.

In some variations, the agent may be subcutaneously injected into the patient. Using the devices and methods described herein, the amount of the agent injected may be as small as about 0.001 cc to about 0.01 cc. For example, a device may be used to deliver a drop of the agent to an acupuncture point. In other variations, a larger dose of the agent may be delivered to the acupuncture point. The larger dose may be, for example, about 1.0 cc or about 2.0 cc.

3) Medical Conditions Alleviated

The agents described herein may be used to treat any medical condition that may benefit from acupuncture or acupressure therapy. The method may also be effective in treating visceral, somatic, inflammatory and neuropathic pain, both acute and chronic, as well as muscle pain and stiffness, and joint pain and stiffness. Examples include joint, muscle, and tendon pain, joint, muscle, and tendon immobility, neuropathies, muscle spasms, osteoarthritis, headaches, and autoimmune disorders. For example, the agents may be used to treat medical conditions such as allergic disorders, cardiovascular disorders, gastrointestinal motility disorders, hormonal disorders, immune disorders, nervous disorders, respiratory disorders, skeletal disorders, urogenital disorders, pain, any combination of the foregoing, and the like. Other conditions that may be treated include, but are not limited to, respiratory conditions, circulatory conditions, nervous conditions, endocrinal conditions, obesity, chest tightness, testicular torsion, sialorrhea, indigestion, ulcers, fracture or compression of lumbar vertebrae, or reflex sympathetic dystrophy, inflammatory arthropathy, bursitis, tendinopathy, sprains, arthralgias, degenerative joint disease, spondylosis, TMJ dysfunction, fibromyalgia, muscle stiffness, overuse syndrome, muscle strains, asthma, atelectasis, chronic obstructive pulmonary disease (COPD), wheezing, dyspnea, high blood pressure, headache, neuropathy, polyneuropathy, tension headache, headache, mood disturbance, sleep disturbance, fatigue, hypersomnia, mood disorder, pancreatitis, diabetes, or allergy.

Furthermore, the agent may be used to alleviate any side effect caused by the use of the tissue-piercing member and vice-versa. In some variations, the agent may be used to relieve pain caused by the insertion of the tissue-piercing member into the skin. In other variations, the tissue-piercing member, by virtue of its location at or adjacent to an acupuncture point, may be used to alleviate a side effect caused by the agent. When included in a kit, the agent may be included in a range of doses. Likewise, the kit may include a variety of tissue-piercing members in assorted sizes or configurations. The kits may be designed to target specific medical conditions. The kits may also be packaged such that only the one or more agents are provided, or only one or more tissue-piercing members are provided.

The methods described here may stimulate a plurality of points including acupuncture/acupressure points as well meridians and points overlying inflamed or damaged tendons, tendon insertions, joints, nerves, and soft tissue structures by means of implantation, infusion or injection therein of a pure or admixed fluid, semi-fluid, powder, particle, gas, charge, field, solid, or semi-solid.

It is another object of this invention to stimulate a plurality of points including acupuncture/acupressure points as well meridians and points overlying inflamed or damaged tendons, tendon insertions, joints, nerves, and soft tissue structures by means of external application of magnets, therapeutic creams, pressure or electricity together or separately after implantation, infusion or injection of a pure or admixed fluid, semi-fluid, powder, particle, gas, charge, field, solid, or semi-solid in or around acupuncture/acupressure points or meridians.

### IV. KITS

The agents and devices may be provided in kits. In general, the kits will include one or more devices, one or more agents that treat a medical condition or relieve pain, and instructions for use. The included agents may be of the same dosage form or different dosage forms. The agents may be packaged in a vial, packet, blister, or the like. The kits may also provide each agent as separately packaged units. Instructions may be in written or pictograph form, or can be on recorded media including audio tape, audio CD, video tape, DVD, CD-ROM, or the like. The instructions may include instruction for locating one or more acupuncture points.

In some variations, the device or tissue-piercing member included in the kit may be provided with the agent in a single dosage form. In other variations, the device or tissue-piercing member may be included in the kit in a dosage form separate from dosage form including the agent. The kits may also be formed to only include agents in any suitable dose. In some instances, a range of doses may be provided.

The kits may be designed to target specific medical conditions. In one variation, the kit is designed for use with osteoarthritic pain. Such a kit may include one or more devices and one or more agents for relieving joint pain. For example, the osteoarthritis kit may provide acupuncture needle(s), and an agent(s) including bupivicaine.

In some variations, the kits may also include electrodes, a TENS unit, LEDs or lights, ultrasound probes, detachable magnets, magnetized needles, a heat source, and/or a negative pressure vacuum. In another variation, a kit is provided which includes a model with the location of acupuncture points, items of clothing in different sizes such as shirts, pants, socks, gloves with holes cut out corresponding to important injection points, a body point chart or booklet with instructions whereto inject for particular conditions, glasses with a 3-D representation of the different injection points superimposable over the patient's body, therapeutic magnets, an electrotherapy or electrokinetic unit, LEDs or lights of different wavelengths, phono or iontophoretic unit, herbs, incense, hypnotic tapes or music, an external device for applying pressure over the site of injection, and/or cups or jars for cupping therapy.

### V. EXAMPLES

The present invention will be further understood by reference to the following non-limiting examples.

### Example 1

### Treatment of Patients with Arthritis

### Injection of Lidocaine

(a) Treatment of a Patient with Rheumatoid Arthritis

(i) A patient presented with a 5/10 pain from severe rheumatoid arthritis. 2 cc lidocaine injected bilaterally and subcutaneously over glenohumeral joints in the vicinity of acupuncture/acupressure point Lu 1 resulted in disappearance of all pain and return of mobility. The patient stated: “It's like a miracle.”

(ii) A patient presented with an 8/10 pain from severe rheumatoid arthritis. 0.5 cc saline injected bilaterally and subcutaneously over the glenohumeral joints in the vicinity of acupuncture/acupressure point Lu 1 resulted in disappearance of all pain. Patient stated: “This is incredible. I can't remember when I've been able to move like this.”

(iii) A female patient presented with 8/10 pain from severe rheumatoid arthritis with diffuse joint stiffness, swelling and tenderness. 2 mL 2% lidocaine+0.5 mL triamcinolone was injected bilaterally into the glenohumeral joints with diminishment of the pain to 2/10 which lasted 1 month post injection.

(iv) A patient with severe RA who was injected with lidocaine subcutaneously over the glenohumeral joints bilaterally experienced a resolution of all pain. The patient was remained pain free for 2 weeks.

(b) Treatment of Patients with Seronegative Arthritis

(i) A patient presented with a pain level of 10/10 from cervical strain due to seronegative rheumatoid arthritis. 1 cc lidocaine injected bilaterally subcutaneously over the glenohumeral (GH) joints reduced pain to 4/10. Another 2 cc lidocaine injected bilaterally and subcutaneously over the GH joints reduced pain to 3/10. 2 cc saline injected bilaterally and subcutaneously reduced pain to 2/10.

(ii) A patient presented with a 7.5/10 pain globally from a seronegative arthritis (unspecified). 2 cc lidocaine injected bilaterally and subcutaneously over the GH joints resulted in disappearance of all pain “for the very first time in my life since this arthritis pain started.”

(c) Treatment of Patients with Glenohumeral Arthritis

(i) A patient presented with a 6/10 shoulder pain due to glenohumeral arthritis and 7/10 low back pain due to lumbar stenosis experienced complete pain relief after 2 cc of lidocaine was injected into his glenohumeral joints bilaterally.

(ii) A patient presented with right glenohumeral osteoarthritis 9/10 and 8/10 low back pains due to sacroiliac strain experienced total relief after injection of 2 ccs bilaterally into the glenohumeral joints.

(d) Treatment of a Patient with Radiohumeral Arthritis and Back Pain

A patient with a 9/10 elbow pain due to radiohumeral arthritis and 10/10 mechanical low back pain experienced complete relief after 2 cc injection lidocaine into his glenohumeral joints in the vicinity of acupuncture/acupressure points Lu 1 bilaterally. Pain relief lasted for 3 days.

(e) Treatment of Patients with Subtalar Arthritis

A patient presented with a 5/10 pain from bilateral post-traumatic subtalar arthritis. 2 cc lidocaine injected bilaterally and subcutaneously over the GH joints in the vicinity of acupuncture/acupressure points Lu 1 resulted in disappearance of all pain which lasted for 1 week.

### Example 2

### Treatment of Patients with Back, Shoulder, Leg Pain-Injection of Lidocaine

(a) Treatment of Patients with Back Pain

(i) A patient who presented with a 4/10 low back pain experienced no relief after 2 cc saline was injected into the glenohumeral joints bilaterally in the vicinity of acupuncture point Lu 1.

(ii) A patient who presented with 5/10 back pain due to rupture of the annulus experienced no relief after 2 cc 1% lidocaine was injected into the glenohumeral joints bilaterally.

(iii) A patient who presented with a 6/10 mechanical low back pain experienced about 50% relief to about 3/10 after 2 cc of lidocaine was injected bilaterally into his glenohumeral joints.

(iv) A patient presented initially with 9/10 mechanical low back pain. The pain decreased to a 4/10 after injection of 2 cc bilaterally into the glenohumeral joints.

(v) A patient with an L5 radiculopathy complained of a 6/10 low back pain which was unrelieved by trigger point injections, sacroiliac injections and epidurals. After 2 cc injections of sterile saline into the glenohumeral joints the back pain decreased to 2/10.

(vi) A patient who presented with a 7/10 low back pain due to SI strain reported that his pain decreased to 4/10 after injection of 2 cc lidocaine into his glenohumeral joints bilaterally.

(vii) A patient presented with a 8/10 back pain from lumbar facet arthropathy. 2 cc lidocaine injected bilaterally and subcutaneously over the GH joints resulted in disappearance of all pain.

(viii) A patient presented with an 8/10 back pain with radiation down leg from lumbar radiculopathy. 2 cc lidocaine injected bilaterally and subcutaneously over the GH joints reduced pain to 1/10. Patient stated: “I've taken pills, had nerve blocks and epidurals and this is the first time since my accident that I've gotten this kind of pain relief.”

(ix) A patient with unspecified 6/10 low back pain received bilateral intrarticular injections of steroid and lidocaine giving him total pain relief which lasted a few hours.

(x) A patient with lumbar facet arthropathy who was injected with lidocaine subcutaneously over the GH joints bilaterally with resolution of pain. The patient was still pain free one week later.

(xi) A patient with an 8/10 back pain from lumbar facet arthropathy was injected bilaterally and subcutaneously over the GH joints with 2 cc lidocaine. The pains disappeared completely and the patient stated “It's like a miracle”.

(xii) A patient presented with a 9/10 low back pain and 8/10 shoulder and neck pain. Injection of lidocaine and saline in the center of the sternum at the level of the top of the axillary crease reduced the neck pain to 4/10 but had no effect on the back pain.

(b) Treatment of Patients with Pain Due to Sacroiliac Strain

(i) A patient presented with a 5/10 low back pain due to sacroiliac strain experienced complete relief after injection of 2 cc of normal saline into his glenohumeral joints bilaterally.

(ii) A patient presented with a 4/10 pain due to sacroiliac strain. 2 cc lidocaine injected bilaterally and subcutaneously over the GH joints resulted in disappearance of all pain. Patient also stated she felt more awake, alert and had a sense of well-being.

(c) Treatment of Patients with Pain Due to Degenerative Joint Disease

(i) A patient presented with a 5/10 neck pain due to degenerative joint disease felt her pain decrease to 3/10 after prolonged mechanical pressure with the fingers was placed over her glenohumeral joints bilaterally. This effect lasted for about 1 hour.

(ii) A patient presented with a 4/10 neck pain from cervical degenerative disc disease. 2 cc lidocaine injected bilaterally and subcutaneously over the GH joints reduced pain to 2/10.

(d) Treatment of Patients with Spondylosis and Spinal Stenosis

(i) A patient with hemiparesis due to a Brown-Sequard syndrome from cervical spinal spondylosis reported that his neck, shoulder, and low back pain completely disappeared after injection of 1 cc lidocaine+1 cc triamcinolone bilaterally into his glenohumeral joints. He also reported that although he has foot drop he was able to lift his leg much higher after injection of 1 cc lidocaine+1 cc triamcinolone bilaterally into his glenohumeral joints.

(ii) A patient with neck pain secondary to cervical spinal stenosis and a past history of an aneurysmal repair reported that her pain completely disappeared in the office.

(iii) A patient presented with a 9/10 pain globally from cervical spondylotic myelopathy. Injection of multiple acupuncture/acupressure points with lidocaine and saline had no effect.

(e) Treatment of Patients with Post-Surgical Pain

(i) A woman presents with 6/10 postsurgical pharyngeal pain after uvulopalatopharyngoplasty for severe sleep apnea. 2 mL 2% lidocaine+0.5 mL triamcinolone was injected bilaterally into the glenohumeral joints with complete resolution of the pain which continued to last 2 days post injection.

(ii) A woman presented with 8/10 postsurgical chest wall pain after a cardiac bypass surgery. 2 mL 2% lidocaine+0.5 ml triamcinolone was injected bilaterally into the glenohumeral joints with complete resolution of the pain which continued to last 2 days post injection.

### Example 3

### Treatment of Patients with Multiple Indications-Injection of Lidocaine

(i) A patient presented with a 6/10 shoulder pain due to bilateral rotator cuff tendinopathy and 6/10 low back pain secondary to lumbar spinal stenosis experienced about 50% relief to 3/10 after 2 cc injection lidocaine bilaterally into the glenohumeral joints.

(ii) Patient with rotator cuff tendinopathy causing 6/10 right shoulder pain and 4/10 low back pain due to SI strain decreased to a 2/10 in his back and a 2/10 in his shoulder after bilateral injection of 2 cc normal saline into the glenohumeral joints bilaterally. After 2 additional ccs of normal saline was injected bilaterally into the glenohumeral joints, all pain in the back and shoulders disappeared.

(iii) A patient presented with a 6/10 bilateral shoulder pain due to glenohumeral arthritis and 6/10 low back pain due to lumbar spinal stenosis decreased to 1/10 shoulder pain and 2/10 low back pain after injection of 4 ccs of lidocaine into her glenohumeral joints bilaterally. She also reported that she felt more awake, had a better mood, and could breathe easier.

(iv) A patient presented with an 8/10 lower leg pain making any ambulation impossible due to a combination of SI strain, lumbar spinal stenosis, and vascular insufficiency decreased to a 6/10 after injection of 2 cc triamcinolone+2 cc lidocaine into his glenohumeral joints bilaterally. Another 1 cc triamcinolone+1 cc lidocaine was injected subcutaneously over the glenohumeral joint and the patient's pain decreased to a 4/10. In addition, he was also able to start walking without any help, felt more vibrant, had a better mood, and could breathe more easily.

(v) A patient presented with a 3/10 knee pain due to osteoarthritis, 5/10 shoulder pain due to rotator cuff tendinitis and 8/10 hip pain due to hip flexion contractures decreased to 0/10 in her knee and shoulders while the hip pain decreased to a 5/10 after bilateral injection of 1 cc triamcinolone+1 cc lidocaine into her glenohumeral joints bilaterally.

(vi) A patient presented with a 6/10 neck pain from degenerative joint disease (DDD), 5/10 mechanical back pain, and 5/10 pain due to osteoarthritis of the knees. After subcutaneous injection of 2 cc sterile saline over the anterior shoulder bilaterally in the vicinity of the glenoumeral joints all pain disappeared. In addition, he reported that he felt more alert and could breathe more easily.

(vii) A patient presented with a 9/10 shoulder/neck pain due to rotator cuff tendinopathy and glenohumeral arthritis, 9/10 low back pain with radiation down his legs due to lumbar radiculopathy. After subcutaneous injection of 2 cc sterile normal saline over the anterior shoulder bilateraly in the vicinity of the glenohumeral joints the pain in the back and the shoulder/neck decreased to a 5/10. After 2 cc injections of sterile normal saline into the glenohumeral joints the pain in the neck/shoulders and shoulders decreased to a 3. An additional 2 cc of saline was placed into the right glenohumeral joint and the pain in the shoulders disappeared while the pain in the low back decreased to a 2.

(viii) A patient with diffuse arthralgias secondary to inflammatory bowel disease (ulcerative colitis) presented with a 9/10 bilateral shoulder pain, 9/10 knee pain and 9/10 low back pain. After injection of 2 cc saline into the glenohumeral joints the pain in the shoulders decreased to a 2 while the pain in the low back decreased to a 7. An additional 2 cc of saline was injected bilaterally into the glenohumeral joints and the back pain decreased to a 5.5 while the knee pain decreased to a 7.

(ix) A patient presented with cervical stenosis, with a pain level of 8/10 and lumbosacral strain with a pain level of 7/10 experienced complete relief after triamcinolone only was injected into her glenohumeral joints bilaterally.

(x) A patient presented with a 5/10 neck pain, 5/10 mechanical low back pain and 3/10 foot pain due to Morton's neuroma was injected with 1 cc triamcinolone+1 cc lidocaine with complete resolution of his symptoms in the office. Three days later, he reported that his neck pain has increased now to a 3/10 which is still less than the pain levels he had before he came to the office, his back pain is still gone but that the pain from the Morton's neuroma had completely returned.

(xi) A patient presented with a 10/10 post surgical pain from tracheostomy and a 6/10 pain from coccygodynia. With subcutaneous injection of 2 cc lidocaine bilaterally over the glenohumeral joint (GH) pain decreased to 2/10 on the right side of the body. Another 2 cc lidocaine over only the left GH joint resulted in disappearance of all pain.

(xii) Additional treatments where 2 cc of lidocaine was injected and observations are shown in the table below.

| | Pain level | |
|---|---|---|
| Patient Condition | Before | After |
| Peptic Ulcer with gnawing epigastric pain | 8/10 | 0 |
| Multiple Sclerois with pain in legs | 7/10 | 0 |
| Lumbar radiculopathy, herniated disk and scatia | 8/10 | 0 |
| Knee pain | 5/10 | 0 |
| Chronic pelvic pain of non menstrual origin | 6/10 | 0 |
| Cervical spondylotic myelopathy | 9/10 | 1/10 |
| Reflex sympathetic dystophy | 6/10 | 0 |
| Plantar Fasciitis | 6/10 | 0 |
| Dermatomyositis | 6/10 | 2/10 |

### Example 4

### Treatments with Saline

(i) A patient presented with a 2/10 rotator cuff tendinopathy. 0.5 cc saline injected bilaterally and subcutaneously over GH joints resulted in disappearance of all pain. Patient also stated he felt more awake, alert and had a sense of well-being.

(ii) 5/10 sacroiliac strain. No relief when needles were inserted subcutaneously over the GH joints. 0.5 cc saline injected subcutaneously and bilaterally over GH joints resulted in disappearance of all pain. Patient stated that she felt more relaxed, calmer, could breathe easier, and had a heightened sense of well-being.

(iii) A drug-seeking patient with likely vertebral compression fracture noted no pain relief with 0.5 cc saline injected bilaterally and subcutaneously over GH joints but did report feeling “more agile.”

(iv) A patient presented with a 6/10 pain globally from severe systemic lupus erthymatosis (SLE). 0.5 cc saline injected bilaterally and subcutaneously over GH joints resulted in disappearance of all pain to the patient's amazement who stated: “This has never ever happened before.” She also reported feeling much more awake, energized and had a sense of “contentment and well-being.”

(v) A patient presented with a 7/10 pain right medial knee pain from pes anserine bursitis. 0.5 cc saline injected subcutaneously over the left GH joint resulted in the disappearance of all pain (unilateral injection of steroid, saline, or lidocaine over the GH joint seems to provide analgesia on the opposite side of the body).

### Example 5

### Treatment with Viscous Triglycerides

(A) Injection of Caprylic/Capric Triglycerides

(i) A patient with an 8/10 neck and shoulder pain from cervical stenosis and 6/10 mechanical low back pain. Injection of 0.1 cc of viscous caprylic/capric triglycerides subcutaneously over the glenohumeral joints bilaterally resulted in alleviation of all pain; patient was still pain free about 24 hours later.

(ii) A patient with 8/10 shoulder pain from rotator cuff tear and 9/10 ankle pain due to post-traumatic osteoarthritis and subtalar joint degeneration. 0.1 cc viscous caprylic/capric triglycerides was injected subcutaneously under the medial and lateral tibial tubercles bilaterally, over the glenohumeral joints bilaterally, over the superomedial angle of the scapula bilaterally and three finger breaths above the medial malleolus bilaterally with alleviation of all pain for 45 minutes before the effect completely wore off.

(iii) A patient with a 7/10 rotator cuff tendinopathy and right patellofemoral pain. Injection of 0.1 cc viscous caprylic/capric triglycerides subcutaneously over the glenohumeral joints and under the medial tibial plateau bilaterally resulted in alleviation of all pain which continued to last almost 24 hours later. The patient reported “This is the first good night of sleep I have had in years because I was pain free.”

(iv) A patient with a 4.6/10 right patellofemoral pain. Injection of 0.1 cc viscous caprylic/capric triglycerides subcutaneously under the medial tibial plateau resulted in alleviation of all pain; patient was still pain free almost 24 hours later.

(v) A patient with an 8/10 low back pain and hand/wrist pain from psoriatic arthritis. 0.1 cc injection of viscous caprylic/capric triglycerides subcutaneously over the glenohumeral joints and under the medial and lateral tibial tubercles bilaterally resulted in alleviation of all pain. The patient reported that 1 day later she has a 50% return of the pain but still feels good enough where she can “get out and move around.”

(B) Injection with a Combination of Lidocaine and Capric/Capryllic Triglycerides

(i) A patient presented with a 9/10 pain from severe bilateral end-stage osteoarthritis of knees. No change was observed with multiple injections of 0.1 cc capric/capryllic triglycerides+1 cc 2% lidocaine into multiple acupuncture/acupressure points.

(ii) A patient presented with a 6/10 shoulder and neck pain from bilateral rotator cuff tears. Injection of 0.1 cc capric/capryllic triglycerides+1 cc lidocaine over the anterior shoulders bilaterally completely eliminated all pain.

(iii) A patient presented with a7/10 pain from C7 radiculitis. No change was observed with injections of 0.1 cc capric/capryllic triglycerides+1 cc 1% lidocaine over anterior shoulders.

(iv) A patient presented with an 8/10 pain from lumbar stenosis and diabetic polyneuropathy. Injections over anterior shoulders, sternum and on the midline about 3 inches below the umbilicus (corresponding to alarm point CV 5) reduced pain to 6/10.

(v) A patient presented with an 8/10 pain globally (cause unknown) with extreme anxiety. Injections over the anterior shoulders and sternum at the level of the top of the axillary crease reduced pain to 5/10 and calmed his anxiety.

(vi) 9.8/10 pain in shoulders and back from glenohumeral arthritis and sacroiliac strain. Injections of 0.1 cc capric/capryllic triglycerides+1 cc lidocaine over the anterior shoulders and sternum reduced pain to 4.5/10. Results of injections with of 0.1 cc capric/capryllic triglycerides+1 cc lidocaine with follow up results are shown in Table 2.

(vii) A patient presented with a 6/10 pain in shoulders, neck and low back with extreme somnolence. Injections of 0.1 cc capric/capryllic triglycerides+1 cc lidocaine into the anterior shoulders bilaterally and over the midline sternum at the level of the top of the axillary crease reduced pain globally to a 2/10 and resulted in a dramatic awakening. This patient stated that he had received several sessions of acupuncture in the past without any beneficial effect.

Table 2. Injection of 0.1 cc capric/capryllic triglycerides+1 cc lidocaine (C/CTG/L) with follow up results.

| | Pain Level | | |
|---|---|---|---|
| | Before | After | |
| Pain Source | C/CTG/L | C/CTG/L | Follow up Observation |
| Neck and Back | 5/10 | 0 | ^{(a)}Back pain returned 1 hr |
| | | | later, pain free in neck 2 |
| | | | days later |
| Neck and Back | 5/10 | 0 | Pain free 2 days later |
| Back | 6/10 | 0 | Pain returned several |
| | | | hours later |
| Back | 7/10 | 0 | ^{(b)}Pain returned several |
| | | | hours later |
| Rheumatoid | 5/10 | 0 | Pain free 2 days later |
| Arthritis | | | |
| Neck/Shoulder | 6/10 | 0 in | Pain free in shoulder 2 |
| | | shoulder/no | days later |
| | | change in | |
| | | neck | |
| Low back | 8/10 | 0 | Still pain free 2 days later |
| Peptic Ulcer | 10/10 | 0 | ^{(c)}Still pain free 2 days |
| Disease | | | later |

^{(a)}The patient also reported that he vomited several times after the injections.
^{(b)}Patient stated that her shoulders are so extremely sore where she was injected that she can barely lift her arms.
^{(c)}Patient was also taking acid suppressive therapy before and after injection.

(viii) 10/10 pain from cervical osteoarthritis and lateral epicondylitis. Injections of 0.1 cc capric/caprvilic triglycerides+1 cc lidocaine over the anterior shoulders and sternum reduced pain to 1/10. Later, corticosteroid+lidocaine doses were injected.

(ix) 8/10 pain from neck and shoulders due to cervical ostearthritis and rotator cuff bursitis. Insertion of needles alone for about 30 seconds over the anterior shoulders had no effect but after injection of 0.1 cc capric/caprvilic triglycerides+1 cc lidocaine, the patient experienced some relief. After injection over sternum patient's pain levels decreased to 1/10.

(x) Extremely somnolent patient almost unarousable due to taking benzodiazepines reported 8/10 low back pain from sacroiliac strain. Injection over anterior shoulders and posteriorly over the tip of the 12th rib completely eliminated all pain and dramatically woke patient up.

### Example 6

### Treatment of Patients with Previous Acupuncture Experience

(i) A patient presented with a 10/10 phantom limb pain and back pain in a double amputee. The patient reported he had tried acupuncture before without success. Injection of lidocaine and saline bilaterally and subcutaneously in the anterior shoulder in the vicinity of Lu 1 reduced pain to 6/10 in his back but did not help his phantom limb pain.

(ii) A patient presented with a 7/10 pain in the low back. Injection of multiple acupuncture/acupressure points with 0.5 cc 2% lidocaine and saline had no effect. This patient has also received acupuncture in the past without success.

(iii) A patient presented with a 9/10 pain due to knee osteoarthritis, cervical stenosis and low back pain from degenerative disc disease. This patient had tried several sessions of acupuncture in the past without any success. Injection of lidocaine and saline in the center of the sternum at the level of the top of axillary crease and in the anterior shoulders and 2 inches lateral to the navel on the left side in the vicinity of acupuncture/acupressure point CV 17 eliminated all pain.

(iv) A patient presented with an 8/10 neck and back pain. In the past, this patient tried acupuncture which had about a 30% effect. Injection of lidocaine and saline in the anterior shoulder bilaterally and subcutaneously in the area of Lu 1 reduced pain globally to 4/10 so this effect of 50% was an improvement over her previous experience with acupuncture.

(v) A patient presented with an 8/10 neck and back pain from cervical stenosis and S1 radiculopathy, respectively. Injections of 0.1 cc capric/capryllic triglycerides+1 cc lidocaine over the anterior shoulders bilaterally in the area of Lu 1 and sternum at the level of the top of the axillary crease in the area of CV 17 completely eliminated all pain. This patient stated that he had received acupuncture in the past without any effect.

### Example 7

### Injection of Lidocaine and Saline

(i) A patient presented with a 9/10 neck pain due to degenerative disc disease and low back pain from an L5 radiculopathy. Injection of lidocaine and saline in the anterior shoulders bilaterally in the area of Lu 1 eliminated all pain.

(ii) A patient presented with an 8/10 joint pain globally from dermatomyositis. Injection of lidocaine and saline in the center of the sternum at the level of the top of axillary crease in the area of CV 17 and in the anterior shoulders bilaterally in the area of Lu 1 reduced pain to 4/10.

(iii) A patient presented with a 7/10 low back pain from degenerative disc disease. Injection of multiple acupuncture/acupressure points with 1 cc lidocaine and saline barely reduced pain levels to 6/10.

(iv) A patient presented with a 7/10 left sided limb pain from reflex sympathetic dystrophy. Injection of multiple acupuncture/acupressure points with 0.5 cc lidocaine and saline had no effect.

(v) A patient presented with an 8/10 pain from sacroiliac strain. Injection of 0.3 cc lidocaine and saline in the anterior shoulders bilaterally in the area of Lu 1 eliminated all pain.

(vi) A patient presented with a 7/10 groin pain due to inguinal lymphadenopathy. Injection of 1 cc lidocaine and saline in the center of the sternum at the level of the top of axillary crease in the area of CV 17 eliminated all pain.

(vii) A patient presented with a 6/10 pain from bilateral rotator cuff tendinopathy. Injection of 1 cc lidocaine and saline in the anterior shoulders in the area of Lu 1 bilaterally eliminated all pain.

### Example 8

### Treatment with Epinephrine or Restylane at Specific Influential Points

(A) 0.1 cc of Lidocaine HCL 1% and Epinephrine 1:100,000 Injection

(i) A patient presented with a 6/10 left ankle pain from gout. Injection given on the right ankle at GB 39, the “influential point of marrow,” located just above the external malleolus, completely relieved all pain. Patient remained pain-free for 1 month.

(ii) A patient presented with an 8/10 pain and stiffness from Parkinson's disease. Injection bilaterally at GB 39, the “influential point of marrow,” located just above the external malleolus, completely relieved all pain. After 1 day, the pain returned.

(iii) A patient presented with a 5/10 neck pain and stiffness. Injection bilaterally at Lu 1 over the anterior shoulders completely relieved all pain. The patient continued to remain pain-free for over 1 month.

(iv) A patient presented with a 5/10 neck and upper back pain from syringomyelia. Injection bilaterally at BL 11,the “influential point of bone,” two fingers breadth bilateral to C7-T1, and at CV 17 (ren mai), the “influential point of chi,” located two inches above the xiphoid process, completely relieved all pain. Patient remained completely pain-free for 1 month and states that a small amount of the pain has returned.

(v) A patient presented with a 5.8/10 right shoulder pain from rotator cuff tendinopathy. Injection at left Lu 1 over the anterior shoulder completely relieved all pain for 2.5 days.

(vi) A patient presented with a 2/10 shoulder pain from a rotator cuff tendinopathy. Injection bilaterally at BL 11, the “influential point of bone,” two fingers breadth bilateral to C7-T1, reduced pain levels to 1/10. The patient continued to remain at a 1/10 pain level for about 1 month.

(vii) A patient presented with a 10/10 frontal sinus headache which was present for over 1 week. Injection of lidocaine+epinephrine bilaterally over Li 4 (Adjoining Valley), an acupressure point located in the middle of the fleshy mound between the thumb and index finger, completely eliminated the headache. The patient continued to remain headache-free for over 1 month.

(viii) A patient presented with a 4/10 tension headache over the temples and neck with photophobia on which is present 2-3 times a week on average. Injection of lidocaine+epinephrine bilaterally over Li 4 (Adjoining Valley), an acupressure point located in the middle of the fleshy mound between the thumb and index finger, completely eliminated the headache. The patient remained headache free for over 2 weeks. This patient has used acupuncture in the past without success.

(ix) A patient presented with 10/10 pain from bilateral tarsal tunnel syndrome. Injection of 0.1 cc of lidocaine and epinephrine bilaterally over source point K3 has resulted in complete elimination of pain for over 2 months.

(x) A patient with chronic radicular left leg pain 6/10. Injection of 0.1 cc lidocaine and epinephrine in the area of basic acupressure point B60 completely eliminated the pain for over 2 months.

(B) Injection of 0.15 cc of Restylane (Hyaluronic Acid)

(i) A patient presented with a 5/10 pain globally especially in the low back. The patient had never had acupuncture before. Injections at GB 39, the “influential point of marrow,” located just above the external malleolus bilaterally, BL 11, the “influential point of bone,” two fingers breadth bilateral to C7-T1, CV 17 (ren mai), the “influential point of chi,” located two inches above the xiphoid process, GB 34, the “influential point of the sinews,” located bilaterally just in front of the tibial tuberosity, and Lu 1 bilaterally over the anterior shoulders, reduced pain to 1/10. The patient stated “this is the best pain relief that I have ever received.” He also noted that he felt much more awake and alert. The patient continued to remain at a 1/10 six days later.

(ii) A patient presented with a 5/10 pain in the mid back who had never had acupuncture before. Injections at Lu 1, BL 17, the “influential point of blood,” two fingers breadth bilateral to T5-6, and CV 17 reduced pain to a 1/10. The patient's pain level was a 2/10 6 days later, with the patient stating “I feel very very good.”

(iii) A patient presented with a 10/10 pain in an amputee with a compound fracture of his femur. He had received acupuncture in the past but it did not work for him. Injections in multiple sites relieved pain to a 9/10 for about 5 minutes then all pain returned.

(c) Treatments in Combination with Magnets

(i) A patient presented with common peroneal neuropathy with a pain level of 9/10. Subcutaneous injection of lidocaine with epinephrine and subcutaneous injection of 0.001 iron dextran, containing 50 mg of iron per mL, near GB34 Yanglingquan in a depression anterior and inferior to the head of the fibula, resulted in pain relief for 3 days at which point all pain returned. At this point he put a magnet over the site of injection and the pain was reduced to a baseline 2/10 at rest with exacerbations according to his activity level. Patient's pain levels, when he is at rest, continued to remain at about a 2/10 over 2 months later.

(ii) Patient with 9/10 back pain from sacroilitis. Injection of 0.1 cc lidocaine 2% bilaterally and subcutaneously over the sacroiliac joints in the area of potent gates of chi point B 47 and acupressure point B 23 resulted in disappearance of all back pain. The pain started to come back 2 days later and the patient put therapeutic magnets over the sites of injection at which point the pain again disappeared. He continued to remain pain-free 1 week later,

### Example 9

### Injection of Lidocaine or Lidocaine with Epinephrine for Non-Painful Conditions

(i) Patient with excessive drooling (sialorrhea). Injection of 0.1 cc lidocaine 2% in the vicinity of Li 4 in the area between the thumb and index finger resulted in cessation of drooling which lasted for 2 weeks.

(ii) Patient with severe emotional upset after the sudden death of her cat. Injection of 0.1 cc of 1% lidocaine+epinephrine in a 1:1,000,000 dilution resulted in a noticeable calming effect which allowed patient to sleep through the night.

(iii) Patient with narcolepsy falling asleep in the office. Injection of 0.1 cc 2% lidocaine in the vicinity of Li 4 in the area between the thumb and index finger noticeably woke patient up. The effect lasted throughout the day.

(iv) Patient with extreme nausea. Injection of 0.1 cc of 1% lidocaine+epinephrine in a 1:1000000 dilution in the vicinity of Pc 6 on the palmar side of the wrist about 2 thumb widths above the wrist crease and in the center of the arm immediately quelled the nausea.

(v) Patient with sneezing and congestion due to allergies. Injection of 0.1 cc of 1% lidocaine+epinephrine in a 1:1,000,000 dilution in the vicinity of B10 about ½ inch below the base of the skull and ½ inch outward from the spine led to an immediate feeling of decongestion.

### Example 10

### Injection of Lidocaine or Lidocaine with Epinephrine Over Inflamed and Damaged Joints, Nerves, Bursae and Entheses

(i) 10/10 pain from sacroilitis. Injection of 0.1 cc 2% lidocaine subcutaneously over the sacroiliac joints resulted in immediate pain cessation and return to full mobility. The patient stated: “I feel like dancing.” Afterwards he put magnets over the two injection sites. Pain relief was still complete 10 days later.

(ii) 5/10 pain from trochanteric bursitis. Injection bilaterally and subcutaneously of 0.1 cc 2% lidocaine over the trochanteric bursae led to 3 weeks of complete pain resolution.

(iii) 7/10 pain from Morton's neuroma. Injection of 0.1 cc 2% lidocaine subcutaneously on the dorsal foot surface between the metatarsal heads resulted in complete resolution of symptoms for 2 weeks.

(iv) 5/10 pain from lateral epicondylitis. Injection of 0.1 cc 2% lidocaine subcutaneously over the lateral epicondyle led to complete pain relief for 1 day.

### Example 11

### Injection of Lidocaine or Lidocaine with Epinephrine in the Contralateral Limb

(i) Patient with 10/10 pain from subtalar arthritis in the right foot. Injection of 0.1 cc of 1% lidocaine+epinephrine in a 1:1,000,000 dilution in the vicinity of Kd 3 on the inside of the medial left ankle resulted in immediate pain relief on the right ankle.

(ii) Patient with 6/10 left medial knee pain. Injection of 0.1 cc of 1% lidocaine+epinephrine in a 1:1,000,000 dilution in the right knee in the vicinity of Sp 9 located on the medial knee below the tibia immediately eliminated all pain in the left knee.

(iii) Patient with 3/10 bilateral heel and calf pain from plantar fascitis. Injection of 0.1 cc of 1% lidocaine+epinephrine in a 1:1,000,000 dilution in the vicinity of Kd 3 on the inside of the medial left ankle resulted in immediate pain relief on the right heel and calf but not on the left. The patient is still pain-free on the right side 4 weeks later.

1. A device for acupuncture therapy, the device comprising:
a tissue-piercing member, wherein the tissue-piercing member is configured to deliver an agent subcutaneously; and
a reservoir for the agent.

2. The device of claim 1, further comprising a housing configured to support a proximal end of the tissue-piercing member and the reservoir.

3. The device of claim 2, wherein the housing is configured to deliver a predetermined amount of the agent from the reservoir to the tissue-piercing member.

4. The device of claim 1, wherein the tissue-piercing member comprises a malleable portion.

5. The device of claim 1, wherein a distal end of the reservoir comprises a tissue-piercing member.

6. The device of claim 1, wherein the tissue-piercing member comprises a acupuncture needle.

7. The device of claim 1, wherein the reservoir is removably coupled to the tissue-piercing member.

8. The device of claim 1, wherein the reservoir is frangible.

9. The device of claim 1, wherein the tissue-piercing member comprises a lumen, wherein an interior surface of the lumen is coated with the agent.

10. The device of claim 1, wherein the agent comprises lidocaine.

11. The device of claim 1, further comprising a second reservoir in fluid communication with the tissue-piercing member and containing a second agent.

12. The device of claim 1, further comprising a pump configured to deliver the agent.

13. The device of claim 12, wherein the pump is configured to deliver the agent periodically.

14. The device of claim 12, wherein the pump is configured to deliver the agent continuously.

15. The device of claim 12, wherein the pump is configured to deliver the agent based on a user input.

16. A device for acupuncture therapy comprising:
a tissue-piercing member configured to deliver an agent when advanced into tissue at or adjacent to an acupuncture point, wherein the tissue-piercing member is coated or impregnated with the agent.

17. The device of claim 16, wherein the tissue-piercing member is spray-coated with the agent.

18. The device of claim 16, wherein the tissue-piercing member is dip-coated with the agent.

19. The device of claim 16, wherein the tissue-piercing member is impregnated with the agent.

20. The device of claim 16, wherein the tissue-piercing member comprises an acupuncture needle.

21. A method for performing acupuncture comprising:
inserting a tissue-piercing member into tissue at or adjacent to an acupuncture point; and
delivering an agent into the tissue.

22. The method of claim 21, wherein delivering the agent comprises pre-treating the tissue by topically administering an agent.

23. The method of claim 22, wherein delivering the agent further comprises passing the tissue-piercing member through the agent at the tissue.

24. The method of claim 21, wherein delivering the agent comprises coating the tissue-piercing member with the agent.

25. The method of claim 21, wherein the tissue-piercing member is coupled to an implantable pump in fluid communication with a reservoir.

26. The method of claim 21, wherein the agent is delivered using a syringe coupled to the tissue-piercing member.

27. The method of claim 21, wherein the tissue-piercing member comprises an acupuncture needle.

28. The method of claim 21, wherein the agent is a liquid, a solid, or a semisolid.

29. The method of claim 28, wherein the liquid is a solution or an emulsion.

30. The method of claim 28, wherein the solid is a powder, a suppository, or a patch.

31. The method of claim 28, wherein the semi-solid is a cream, a lotion, an ointment, or a gel.

32. The method of claim 21, wherein the agent is configured for sustained release.

33. The method of claim 21, wherein the agent is used to treat a joint condition.

34. The method of claim 33, wherein the joint condition is inflammatory arthropathy, bursitis, tendinopathy, sprains, arthralgias, osteoarthritis, degenerative joint disease, spondylosis, TMJ dysfunction, or fibromyalgia.

35. The method of claim 21, wherein the agent is used to treat a muscular condition.

36. The method of claim 35, wherein the muscular condition is muscle stiffness, overuse syndrome, or muscle strains.

37. The method of claim 21, wherein the agent is used to treat a respiratory condition.

38. The method of claim 37, wherein the respiratory condition is asthma, atelectasis, chronic obstructive pulmonary disease, wheezing, or dyspnea.

39. The method of claim 21, wherein the agent is used to treat a circulatory condition.

40. The method of claim 39, wherein the circulatory condition is high blood pressure or headache.

41. The method of claim 21, wherein the agent is used to treat a nervous condition.

42. The method of claim 41, wherein the nervous condition is neuropathy, polyneuropathy, tension headache, headache, mood disturbance, sleep disturbance, fatigue, hypersomnia, or mood disorder.

43. The method of claim 21, wherein the agent is used to treat an endocrinal condition.

44. The method of claim 43, wherein the endocrinal condition is pancreatitis, diabetes, or allergy.

45. The method of claim 21, wherein the agent is used to treat obesity, pain, chest tightness, testicular torsion, sialorrhea, indigestion, ulcers, fracture or compression of lumbar vertebrae, or reflex sympathetic dystrophy.

46. The method of claim 21, wherein the agent comprises anti-atherosclerotic agents, anti-psoriatics, antispasmodics, muscle relaxants, muscle contractants, histamines, antipyretics, analgesics, antihypertensives, anticoagulants, procoagulants, cholesterol-reducing agents, anticonvulsants, cognitive enhancers, cholinergics, anti-cholinergics, anti-Alzheimer's disease agents, sedatives, anti-Parkinson substances, hypnotics, anti-psychotic substances, antacids, antihistamines, antidiabetics, contraceptives, sympathomimetics, coenzymes, adrenergics, adrenergic antagonists, enzyme inhibitors, neurotoxins, neurotransmitters, hormones, anti-ulcer agents, antiflatulents, proton pump inhibitors, antidiarrheals, antipruritics, anti-emetics, antireflux agents, antiobesity agents, autoimmune disorder agents, anti-cancer substances, immunomodulatory factors, diuretics, anti-glaucoma compounds, anti-inflammatory agents, anti-vertigo medications, local anesthetics, ophthalmics, trophic factors, growth factors, nucleic acids, anti-infectives, vitamins, minerals, nutritional supplements, lubricants, imaging agents, emulsifying stabilizers, herbs, plant extracts, astringents, combinations, derivatives, or precursors thereof.

47. The method of claim 21, wherein the agent comprises bupivicaine.

48. The method of claim 21, wherein the agent comprises triamcinolone.

49. The method of claim 21, wherein the agent comprises a local anesthetic in combination with epinephrine.

50. The method of claim 21, wherein delivering the agent into the tissue comprises delivering about 0.01 cc or less of the agent into the tissue.

51. A method for acupuncture therapy comprising:
subcutaneously inserting an implantable reservoir at or adjacent to an acupuncture point, the implantable reservoir containing an agent.

52. The method of claim 51, wherein the implantable reservoir comprises a wick.

53. A patch for acupuncture therapy comprising:
a plurality of tissue-piercing members; and
an agent in fluid communication with the tissue-piercing members, wherein the patch is configured to adhere to skin.

54. The patch of claim 53, wherein the plurality of tissue-piercing members are configured to deliver the agent.

55. The patch of claim 53, wherein the patch is configured to adhere to the skin using one or more adhesives.

56. A kit comprising:
one or more devices for acupuncture therapy; and
one or more reservoirs for one or more agents.

57. The kit of claim 56, further comprising instructions for use.

58. The kit of claim 57, wherein the instructions for use comprise instructions for selecting an acupuncture point based on a type of pain.

59. The kit of claim 56, wherein the one or more devices for acupuncture therapy are coated with the one or more agents.

60. The kit of claim 56, wherein the reservoir comprises at least one blister containing the one or more agents.
